(19) United States
(12) Patent Application Publication
(10) Pub. No.: US 2017/0327690 A1
(43) Pub. Date: Nov. 16, 2017

(54) CATIONIC BENZOXAZINE DERIVATIVES AND USE IN HAIR DYEING

(71) Applicant: L'OREAL, Paris (FR)

(72) Inventor: Aziz FADLI, Chelles (FR)

(21) Appl. No.: 15/537,084

(22) PCT Filed: Dec. 16, 2015
(86) PCT No.: PCT/EP2015/080052

§ 371 Date: Jun. 16, 2017

(30) Foreign Application Priority Data
Dec. 19, 2014 (FR) 1462865

(51) Int. Cl. C09B 19/00; A61K 8/49; A61Q 5/10

(52) U.S. Cl. CPC C09B 19/00; A61Q 5/10; A61K 8/49; A61K 2800/4324

(57) ABSTRACT

The invention relates to novel cationic benzoxazine-based compounds of formula (I) below, in which •R represents a hydrogen atom or a C₁-C₄ alkyl or C₁-C₄ hydroxyalkyl radical, •—R₂, R₃, R₄ and R₅ independently represent: —a hydrogen atom, —a linear C₁-C₄ or branched C₃-C₄ alkyl radical, optionally substituted with a hydroxyl radical, •L represents a linear or branched, saturated C₁-C₃₀ hydrocarbon-based chain, •CAT+ represents a cationic radical chosen from: —a cationic heterocyclic radical comprising 5 to 8 ring members containing at least one heteroatom chosen from O, N and S, —a tri(hydroxy)(C₁-C₄)alkylammonium radical, —a non-cationic heterocycle comprising 5 or 6 ring members containing at least one heteroatom chosen from O and N, such as pyrrolidine, substituted with a cationic radical, An− represents an anion or a mixture of anions which are organic or inorganic and are cosmetically acceptable.

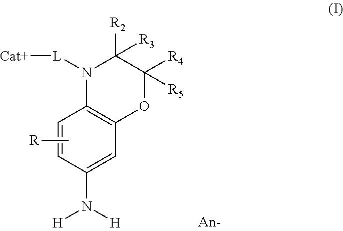

The invention relates to novel cationic benzoxazine-based compounds, to the use thereof for dyeing keratin fibres and also to a dyeing process using these compounds.

It is known practice to dye keratin fibres and in particular human hair with dye compositions containing oxidation dye precursors, which are generally known as oxidation bases, such as ortho- or para-phenylenediamines, ortho- or para-aminophenols and heterocyclic compounds. These oxidation bases are colourless or weakly coloured compounds, which, when combined with oxidizing products, may give rise to coloured compounds via a process of oxidative condensation.

It is also known that the shades obtained with these oxidation bases may be varied by combining them with couplers or colour modifiers, the latter being chosen in particular from aromatic meta-diamines, meta-aminophenols, meta-diphenols and certain heterocyclic compounds such as indole compounds.

The variety of molecules used as oxidation bases and couplers allows a wide range of colours to be obtained.

The “permanent” colouring obtained by means of these couplers and oxidation dyes must moreover satisfy a certain number of requirements. Thus, it should have no toxicological drawbacks, it should allow shades to be obtained in the desired intensity, and it should show good resistance to external agents such as light, bad weather, washing, permanent waving treatments, perspiration and rubbing.

The dyes should also allow grey hair to be covered and, finally, they should be as unselective as possible, i.e. they should produce the smallest possible differences in colour along the same keratin fibre, which in general is differently sensitized (i.e. damaged) between its end and its root.

The aim of the present invention is to obtain hair dyeing compounds that can afford improved dyeing properties in terms of intensity and/or chromaticity and/or selectivity and/or resistance to external agents.

Surprisingly and advantageously, the Applicant has just discovered a new family of oxidation dye precursors constituted of benzoxazine derivatives. These compounds result in a wide range of colours in oxidation dyeing. They in particular make it possible to broaden the colour range, and to obtain powerful, chromatic and sparingly selective colourings in varied shades, which show good resistance to the various external attacking factors to which the hair may be subjected (shampooing, light, sweat or permanent reshaping).

The invention therefore relates to a cationic benzoxazine-based compound of formula (I) below, addition salts thereof, optical isomers, geometrical isomers and tautomers thereof and/or solvates thereof:

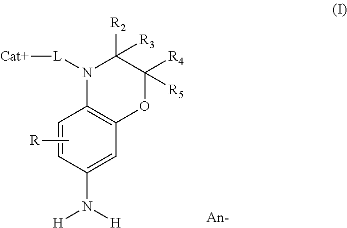

in which
R represents a hydrogen atom or a C₁-C₄ alkyl or C₁-C₄ hydroxyalkyl radical; preferably, R represents a hydrogen atom,
R₂, R₃, R₄ and R₅ independently represent:
   a hydrogen atom,
   a linear C₁-C₄ or branched C₃-C₄ alkyl radical, optionally substituted with a hydroxyl radical,
L represents a linear or branched, saturated C₁-C₃₀ hydrocarbon-based chain, optionally substituted with one or more hydroxyl radicals, optionally interrupted with one or more non-adjacent oxygen atoms or with one or more non-adjacent radicals NR′, R′ denoting a hydrogen atom or a C₁-C₄ alkyl radical,
CAT+ represents a cationic radical chosen from:
   a cationic heterocyclic radical comprising 5 to 8 ring members containing at least one heteroatom chosen from O, N and S, optionally substituted with one or more radicals, which may be identical or different, chosen from linear or branched C₁-C₄ alkyl radicals or linear or branched C₁-C₄ hydroxyalkyl radicals,
   a tri(hydroxy)(C₁-C₄)alkylammonium radical,
   a non-cationic heterocycle comprising 5 or 6 ring members containing at least one heteroatom chosen from O and N, such as pyrrolidine, substituted with a cationic radical chosen from:
imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums, benzoxazoliums and piperidiniums, which are optionally substituted with one or more identical or different radicals chosen from linear or branched C₁-C₄ alkyl radicals and linear or branched C₁-C₄ hydroxyalkyl radicals,
   or a tri(hydroxy)(C₁-C₄)alkylammonium radical,

An⁻ represents an anion or a mixture of anions which are organic or inorganic and are cosmetically acceptable.

A subject of the invention is also the use of a compound of formula (I) below, as defined above, for dyeing keratin fibres, in particular human keratin fibres such as the hair.

A subject of the invention is also a composition, especially a cosmetic composition and in particular a composition for treating keratin fibres such as human keratin fibres and in particular the hair, comprising at least one cationic benzoxazine-based compound of formula (I) described above.

Finally, a subject of the invention is a process for dyeing keratin fibres, in particular the hair, which consists in applying to said fibres a composition comprising a compound of formula (I) described previously.

The compounds of the present invention make it possible in particular to obtain compositions for dyeing keratin fibres that are suitable for use in oxidation dyeing and that make it possible to obtain a hair colouring that has improved dyeing properties in terms of intensity or chromaticity and/or selectivity and/or resistance to external agents such as shampoo, sweat, permanent reshaping and light.

The electroneutrality of the compounds of formula (I) is ensured by an anion or a mixture of anions, labelled An⁻, which are organic or inorganic and are cosmetically acceptable.

An⁻ represents an anion or a mixture of anions chosen, for example, from a halide such as chloride, bromide, fluoride or iodide; a hydroxide; a sulfate; a hydrogen sulfate; an alkylsulfate in which the linear or branched alkyl part is C₁-C₆, such as the methylsulfate or ethylsulfate ion; carbonates and hydrogen carbonates; salts of carboxylic acids, such as formate, acetate, citrate, tartrate and oxalate; alkyl sulfonates for which the linear or branched alkyl part is C₁-C₆, such as the methylsulfonate ion; arylsulfonates for which the aryl part, preferably phenyl, is optionally substituted with one or more C₁-C₄ alkyl radicals, for instance 4-tolylsulfonate; and alkylsulfonyls such as mesylate.

The compound of general formula (I) may be in free form or in the form of salts, such as addition salts with an inorganic acid preferably chosen from hydrochloric acid, hydrobromic acid, sulfuric acid and phosphoric acid or with an organic acid such as, for example, citric acid, succinic acid, tartaric acid, lactic acid, 4-tolylsulfonic acid, benzenesulfonic acid, acetic acid, para-toluenesulfonic acid, formic acid and methanesulfonic acid.

This compound may also be in the form of solvates, for example a hydrate or a solvate of a linear or branched alcohol such as ethanol or isopropanol.

For the purposes of the present invention and unless otherwise indicated:
an “alkyl radical” is a linear or branched C₁-C₂₀ and preferably C₁-C₈ hydrocarbon-based radical;
an “alkenylene radical” is an unsaturated hydrocarbon-based divalent radical as defined previously, which may contain from 1 to 4 conjugated or unconjugated double bonds —C═C—; the alkenylene group particularly contains 1 or 2 unsaturations;
the term “optionally substituted” applied to the alkyl radical means that said alkyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming with the nitrogen atom that bears them a 5- to 8-membered heterocycle, optionally comprising another nitrogen or non-nitrogen heteroatom; v) or a quaternary ammonium group —N⁺R′R″R′″, M⁻ for which R′, R″ and R′″, which may be identical or different, represent a hydrogen atom or a C₁-C₄ alkyl group, or else —N⁺R′R″R′″ forms a heteroaryl such as imidazolium optionally substituted with a C₁-C₄ alkyl group, and M⁻ represents the counterion of the corresponding organic acid, inorganic acid or halide;
an “alkoxy radical” is an alkyl-oxy radical for which the alkyl radical is a linear or branched C₁-C₁₆ and preferentially C₁-C₈ hydrocarbon-based radical;
when the alkoxy group is optionally substituted, this implies that the alkyl group is optionally substituted as defined hereinabove;
the term “at least one” is equivalent to the term “one or more”; and
the term “inclusively” for a range of concentrations means that the limits of that range are included in the defined range.

It should be noted that, in the text hereinbelow, unless otherwise indicated, the limits of a range of values are included in that range.

According to a first subject, the invention thus relates to a compound of formula (I) below, addition salts thereof, optical isomers, geometrical isomers and tautomers thereof and/or solvates thereof:

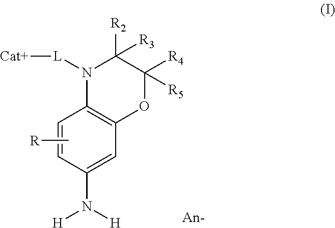

in which
R represents a hydrogen atom or a C₁-C₄ alkyl or C₁-C₄ hydroxyalkyl radical; preferably, R represents a hydrogen atom,
R₂, R₃, R₄ and R₅ independently represent:
   a hydrogen atom,
   a linear C₁-C₄ or branched C₃-C₄ alkyl radical, optionally substituted with a hydroxyl radical,
L represents a linear or branched, saturated C₁-C₃₀ hydrocarbon-based chain, optionally substituted with one or more hydroxyl radicals, optionally interrupted with one or more non-adjacent oxygen atoms or with one or more non-adjacent radicals NR′, R′ denoting a hydrogen atom or a C₁-C₄ alkyl radical,
CAT+ represents a cationic radical chosen from:
   a cationic heterocyclic radical comprising 5 to 8 ring members containing at least one heteroatom chosen from O, N and S, optionally substituted with one or more radicals, which may be identical or different, chosen from linear or branched C₁-C₄ alkyl radicals or linear or branched C₁-C₄ hydroxyalkyl radicals,
   a tri(hydroxy)(C₁-C₄)alkylammonium radical,
   a non-cationic heterocycle comprising 5 or 6 ring members containing a heteroatom chosen from O and N, such as pyrrolidine, substituted with a cationic radical chosen from:
   imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums, benzoxazoliums and piperidiniums, which are optionally substituted with one or more identical or different radicals chosen from linear or branched C₁-C₄ alkyl radicals and linear or branched C₁-C₄ hydroxyalkyl radicals, or
   a tri(hydroxy)(C₁-C₄)alkylammonium radical.

Examples of a cationic heterocycle comprising 5 to 8 ring members containing at least one heteroatom chosen from O, N and S that may be mentioned include imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums, benzoxazoliums and piperidiniums.

By way of tri(hydroxy)(C₁-C₄)alkylammonium radical, mention may be made of trimethylammonium, triethylammonium, dimethylethylammonium, diethylmethylammonium, diisopropylmethylammonium, diethylpropylammonium, 2-hydroxyethyldiethylammonium, di-beta-hydroxyethylmethylammonium, tri-beta-hydroxyethylammonium, dimethylhydroxyethylammonium, 2-hydroxyethyldimethylammonium and 2-hydroxyethylmethylethylammonium.

Preferably, R, R₂, R₃, R₄ and R₅ independently represent a hydrogen atom or a linear C₁-C₄ or branched C₃-C₄ alkyl radical.

Preferably still, R, R₂, R₃, R₄ and R₅ represent a hydrogen atom.

Preferably, L represents a linear or branched, saturated C₁-C₂₀, preferably C₁-C₁₀, hydrocarbon-based chain, optionally substituted with one or more hydroxyl radicals, optionally interrupted with one or two non-adjacent oxygen atoms or with one or two non-adjacent radicals NR′, R′ denoting a hydrogen atom or a C₁-C₄ alkyl radical.

Even more preferably, L represents a linear, saturated C₁-C₁₀ hydrocarbon-based chain, optionally interrupted with an oxygen atom and/or a radical NR′, R′ denoting a hydrogen atom or a C₁-C₄ alkyl radical, preferably a hydrogen atom.

According to one preferred embodiment, L represents a group.

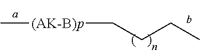

in which:
B represents an oxygen atom or a radical NR′ in which R′ represents a hydrogen atom or a C₁-C₄ alkyl radical,
n represents an integer between 1 and 5, preferably n=1 or 2,
AK represents a linear or branched, saturated C₁-C₁₀ hydrocarbon-based chain which is optionally substituted with one or more hydroxyl radicals, and
p=0, 1 or 2, preferably, p=0 or 1, when p is equal to 2, the radicals B respectively AK are identical or different,
the bond a links the group L to the cationic radical CAT+ and the bond b links the group L to the nitrogen atom of the oxazine ring.

Preferably, R′ represents H or CH₃, and even more preferentially a hydrogen atom. Preferably, AK represents a linear or branched, saturated C₁-C₆ hydrocarbon-based chain, optionally substituted with one or more hydroxyl radicals, preferably still AK represents a linear, saturated C₁-C₄ hydrocarbon-based chain.

Preferably CAT+ represents a cationic radical chosen from:
a cationic heterocyclic radical chosen from imidazoliums, piperaziniums, pyrrolidiniums, morpholiniums and piperidiniums which are optionally substituted with one or more identical or different radicals chosen from linear or branched C₁-C₄ alkyl radicals and linear or branched C₁-C₄ hydroxyalkyl radicals;
a tri(hydroxy)(C₁-C₄)alkylammonium radical chosen from trimethylammonium, triethylammonium, dimethylethylammonium, diethylmethylammonium, diisopropylmethylammonium, diethylpropylammonium, 2-hydroxyethyldimethylammonium, diethylhydroxyethylammonium and 2-hydroxyethyldiethylammonium;
a non-cationic heterocycle comprising 5 or 6 ring members containing a heteroatom chosen from O and N, such as pyrrolidine for example, substituted with a tri(hydroxy)(C₁-C₄)alkylammonium radical, for instance trimethylammonium, triethylammonium, dimethylethylammonium, diethylmethylammonium, diisopropylmethylammonium, diethylpropylammonium, 2-hydroxyethyldiethylammonium, di-beta-hydroxyethylmethylammonium, tri-beta-hydroxyethylammonium, dimethylhydroxyethylammonium or 2-hydroxyethyldimethylammonium, 2-hydroxyethylmethylethylammonium, in particular trimethylammonium or 2-hydroxyethylmethylethylammonium.

Even more preferably, CAT+ represents a cationic radical chosen from:
a cationic heterocyclic radical chosen from imidazoliums, piperaziniums, pyrrolidiniums, morpholiniums and piperidiniums which are optionally substituted with one or more identical or different radicals chosen from linear or branched C₁-C₄ alkyl radicals and linear C₁-C₄ hydroxyalkyl radicals,
a radical chosen from trimethylammonium, triethylammonium, dimethylethylammonium, diethylmethylammonium, diisopropylmethylammonium, 2-hydroxyethylmethylethylammonium 2-hydroxyethyldimethylammonium, di-beta-hydroxyethylmethylammonium, tri-beta-hydroxyethylammonium, dimethylhydroxyethylammonium, and 2-hydroxyethyldimethylammonium, in particular trimethylammonium and 2-hydroxyethylmethylethylammonium,
a pyrrolidine radical substituted with a trimethylammonium radical.

I/ According to a first embodiment of the invention, the compounds of formula (I) are such that:
L represents a linear, saturated C₁-C₆ hydrocarbon-based chain,
R, R₂, R₃, R₄ and R₅ represent a hydrogen atom,
CAT+ represents a cationic radical chosen from:
   a cationic heterocyclic radical chosen from imidazoliums, piperaziniums, pyrrolidiniums, morpholiniums and piperidiniums which are optionally substituted with one or more identical or different linear or branched C₁-C₄ alkyl or linear C₁-C₄ hydroxyalkyl radicals,
   a radical chosen from trimethylammonium, triethylammonium, dimethylethylammonium, diethylmethylammonium, diisopropylmethylammonium, 2-hydroxyethyldimethylammonium, 2-hydroxyethylmethylethylammonium, di-beta-hydroxyethylmethylammonium, tri-beta-hydroxyethylammonium, dimethylhydroxyethylammonium, and 2-hydroxyethyldimethylammonium, in particular trimethylammonium and 2-hydroxyethylmethylethylammonium,
   a pyrrolidine radical substituted with a trimethylammonium radical.

According to this embodiment, the compounds that are particularly preferred are chosen from:

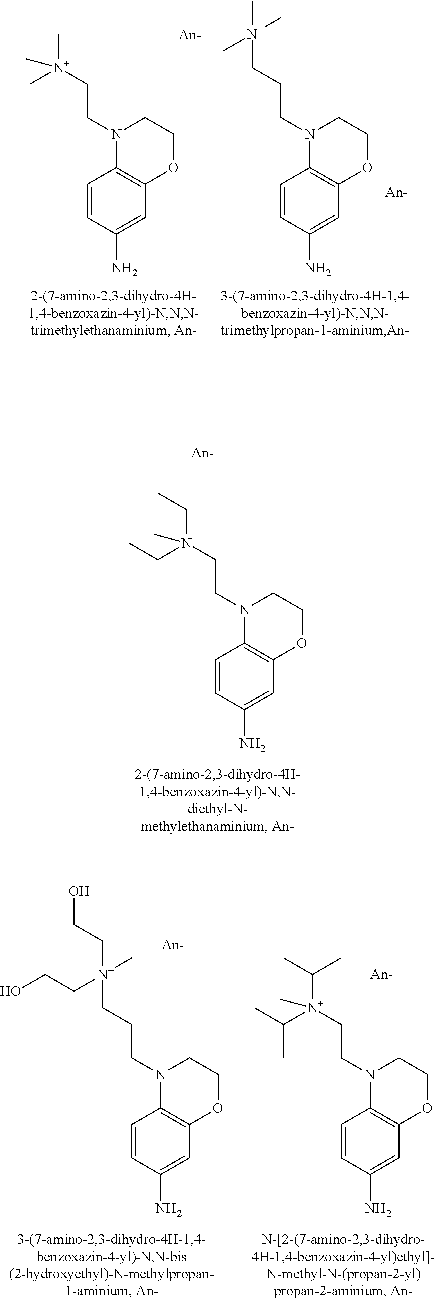 [SMILES: C[N+](C)(C)CCN1CCOc2cc(N)ccc21 ; C[N+](C)(C)CCCN1CCOc2cc(N)ccc21 ; CC[N+](C)(CC)CCN1CCOc2cc(N)ccc21 ; C[N+](CCO)(CCO)CCCN1CCOc2cc(N)ccc21 ; CC(C)[N+](C)(CCN1CCOc2cc(N)ccc21)C(C)C]

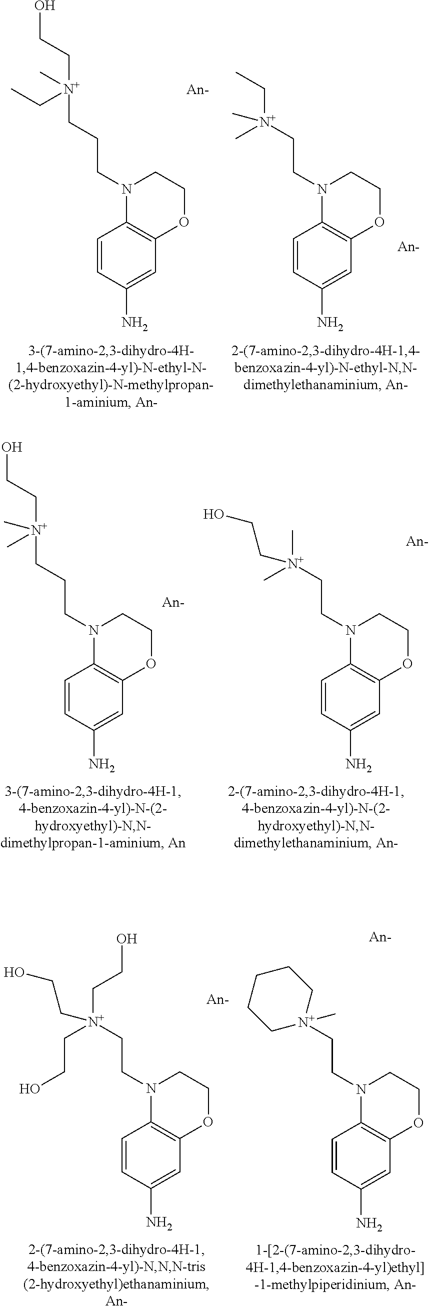 [SMILES: CC[N+](C)(CCO)CCCN1CCOc2cc(N)ccc21 ; CC[N+](C)(C)CCN1CCOc2cc(N)ccc21 ; C[N+](C)(CCO)CCCN1CCOc2cc(N)ccc21 ; C[N+](C)(CCO)CCN1CCOc2cc(N)ccc21 ; Nc1ccc2c(c1)OCCN2CC[N+](CCO)(CCO)CCO ; C[N+]1(CCN2CCOc3cc(N)ccc32)CCCCC1]

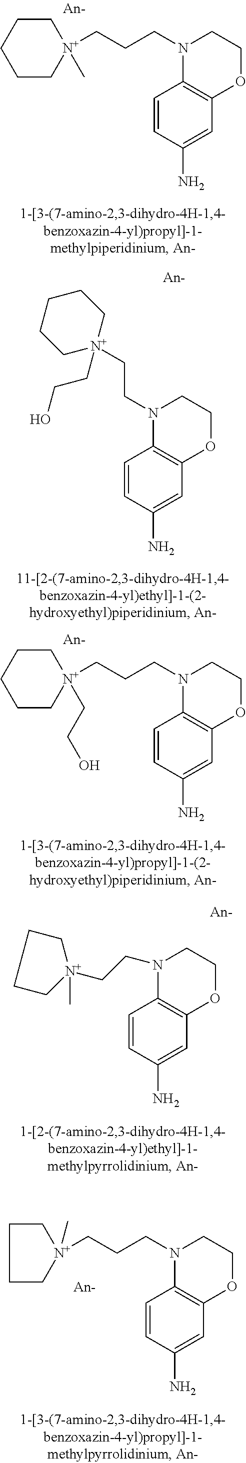 [SMILES: C[N+]1(CCCN2CCOc3cc(N)ccc32)CCCCC1 ; Nc1ccc2c(c1)OCCN2CC[N+]1(CCO)CCCCC1 ; Nc1ccc2c(c1)OCCN2CCC[N+]1(CCO)CCCCC1 ; C[N+]1(CCN2CCOc3cc(N)ccc32)CCCC1 ; C[N+]1(CCCN2CCOc3cc(N)ccc32)CCCC1]

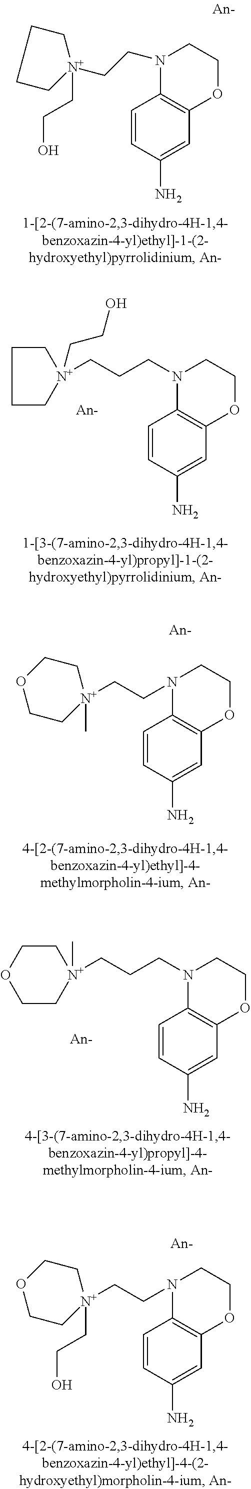 [SMILES: Nc1ccc2c(c1)OCCN2CC[N+]1(CCO)CCCC1 ; Nc1ccc2c(c1)OCCN2CCC[N+]1(CCO)CCCC1 ; C[N+]1(CCN2CCOc3cc(N)ccc32)CCOCC1 ; C[N+]1(CCCN2CCOc3cc(N)ccc32)CCOCC1 ; Nc1ccc2c(c1)OCCN2CC[N+]1(CCO)CCOCC1]

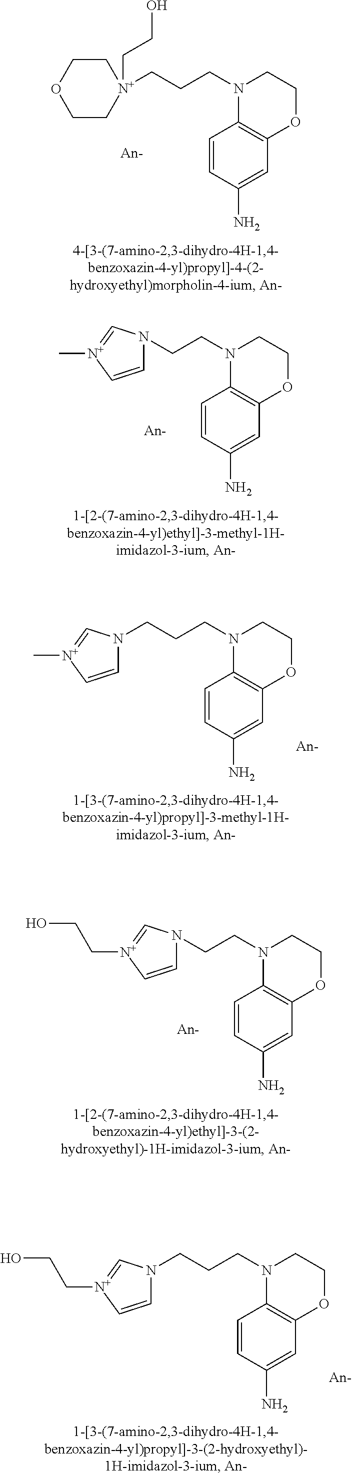 [SMILES: Nc1ccc2c(c1)OCCN2CCC[N+]1(CCO)CCOCC1 ; C[n+]1ccn(CCN2CCOc3cc(N)ccc32)c1 ; C[n+]1ccn(CCCN2CCOc3cc(N)ccc32)c1 ; Nc1ccc2c(c1)OCCN2CCn1cc[n+](CCO)c1 ; Nc1ccc2c(c1)OCCN2CCCn1cc[n+](CCO)c1]

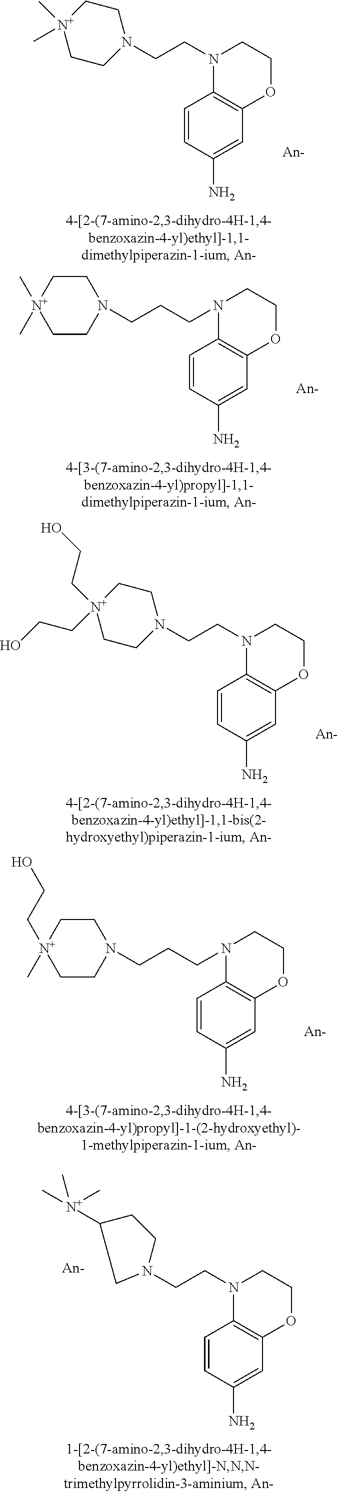 [SMILES: C[N+]1(C)CCN(CCN2CCOc3cc(N)ccc32)CC1 ; C[N+]1(C)CCN(CCCN2CCOc3cc(N)ccc32)CC1 ; Nc1ccc2c(c1)OCCN2CCN1CC[N+](CCO)(CCO)CC1 ; C[N+]1(CCO)CCN(CCCN2CCOc3cc(N)ccc32)CC1 ; C[N+](C)(C)C1CCN(CCN2CCOc3cc(N)ccc32)C1]

and also addition salts thereof, optical isomers, geometrical isomers and tautomers thereof and/or solvates thereof, An− having the meaning given previously.

II/ According to a second embodiment of the invention, the compounds of formula (I) are such that:
L represents a linear C2-C10 alkyl radical, interrupted with an oxygen atom and/or with an —NH group,
R, R2, R3, R4 and R5 represent a hydrogen atom,
CAT+ represents a cationic radical chosen from:
   a cationic heterocyclic radical chosen from imidazoliums, piperaziniums, pyrrolidiniums, morpholiniums and piperidiniums which are optionally substituted with one or more identical or different linear or branched C₁-C₄ alkyl or linear C₁-C₄ hydroxyalkyl radicals,
   a radical chosen from trimethylammonium, triethylammonium, dimethylethylammonium, diethylmethylammonium, diisopropylmethylammonium, 2-hydroxyethyldimethylammonium, 2-hydroxyethylmethylethylammonium, di-beta-hydroxyethylmethylammonium, tri-beta-hydroxyethylammonium, dimethylhydroxyethylammonium, and 2-hydroxyethyldimethylammonium, in particular trimethylammonium and 2-hydroxyethylmethylethylammonium,
   a pyrrolidine radical substituted with a trimethylammonium radical.

According to this second variant, the compounds of formula (I) that are particularly preferred are chosen from:

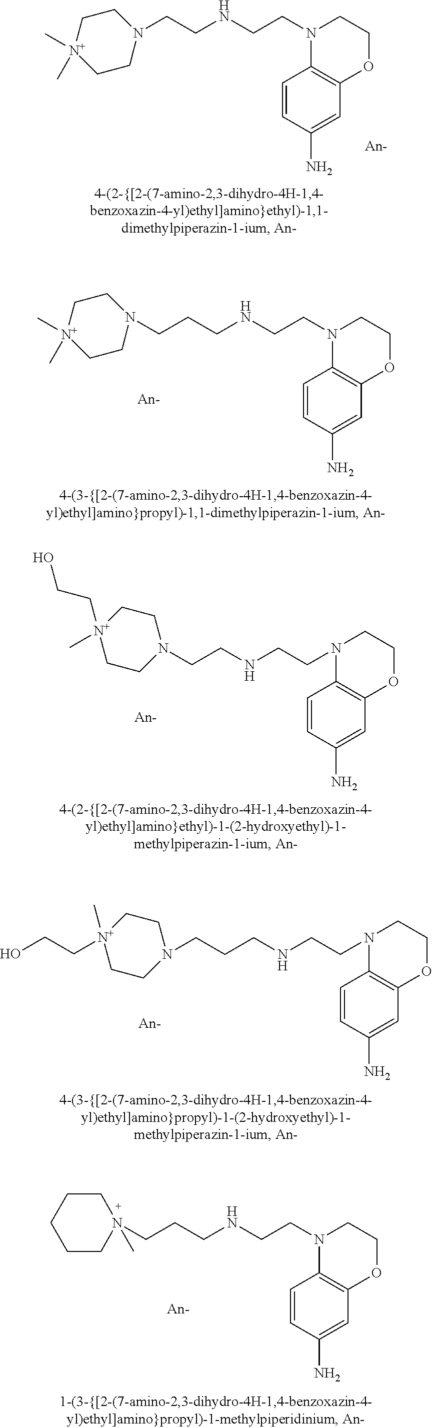

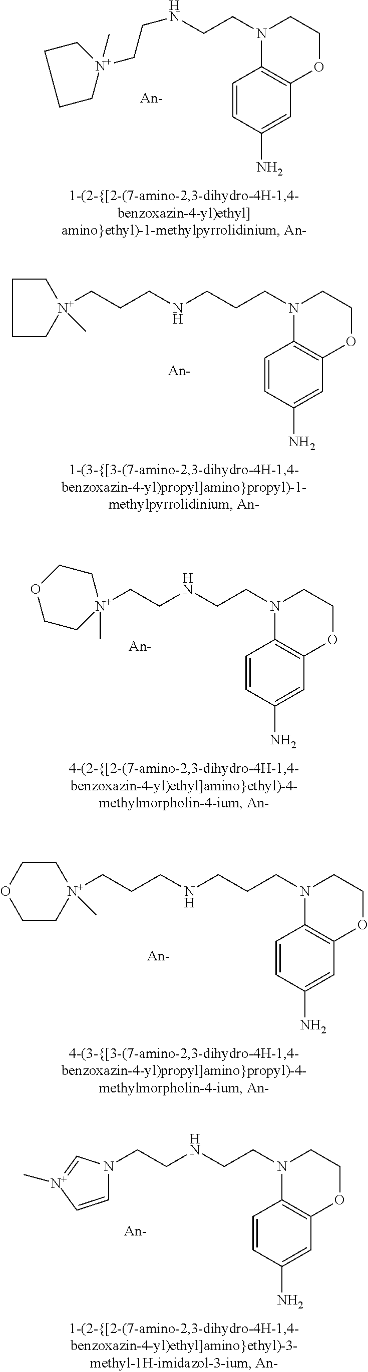 [SMILES: C[N+]1(CCNCCN2CCOc3cc(N)ccc32)CCCC1 ; C[N+]1(CCCNCCCN2CCOc3cc(N)ccc32)CCCC1 ; C[N+]1(CCNCCN2CCOc3cc(N)ccc32)CCOCC1 ; C[N+]1(CCCNCCCN2CCOc3cc(N)ccc32)CCOCC1 ; C[n+]1ccn(CCNCCN2CCOc3cc(N)ccc32)c1]

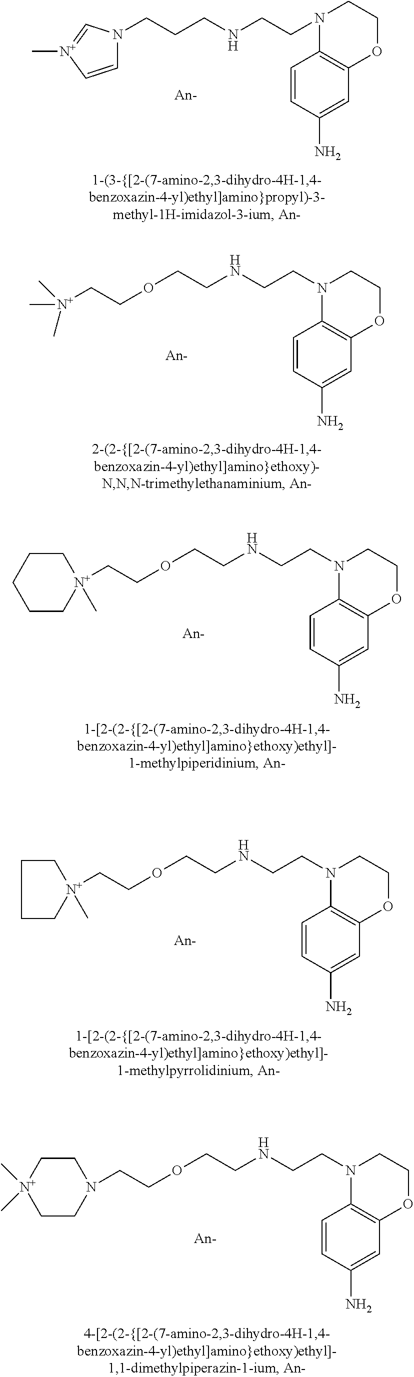 [SMILES: C[n+]1ccn(CCCNCCN2CCOc3cc(N)ccc32)c1 ; C[N+](C)(C)CCOCCNCCN1CCOc2cc(N)ccc21 ; C[N+]1(CCOCCNCCN2CCOc3cc(N)ccc32)CCCCC1 ; C[N+]1(CCOCCNCCN2CCOc3cc(N)ccc32)CCCC1 ; C[N+]1(C)CCN(CCOCCNCCN2CCOc3cc(N)ccc32)CC1]

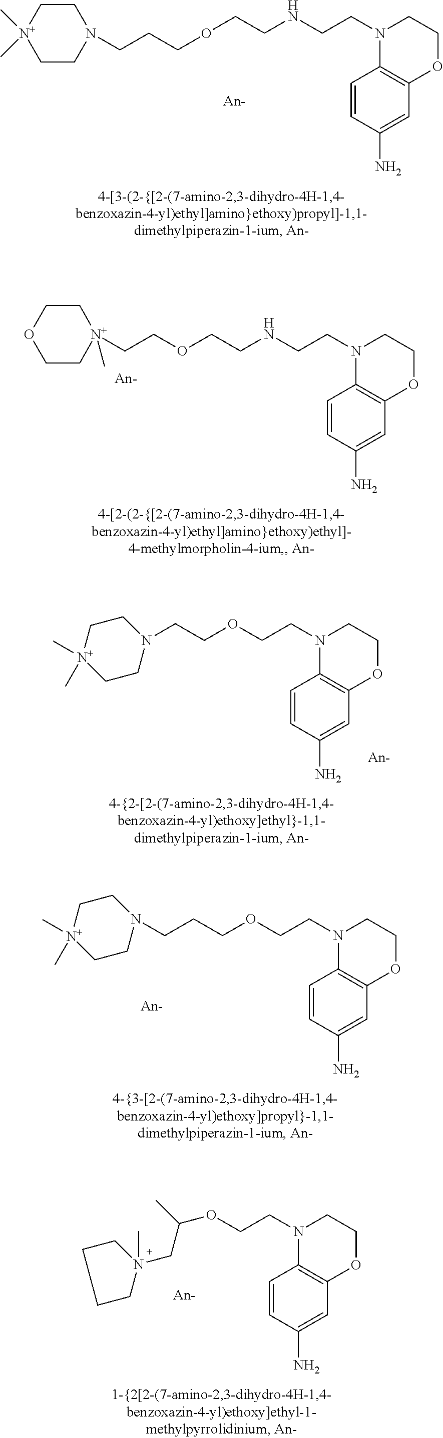

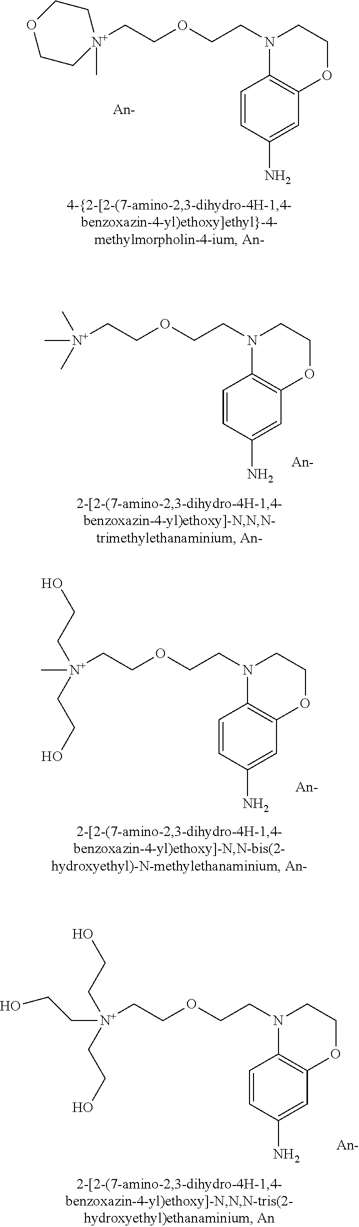 [SMILES: C[N+]1(CCOCCN2CCOc3cc(N)ccc32)CCOCC1 ; C[N+](C)(C)CCOCCN1CCOc2cc(N)ccc21 ; C[N+](CCO)(CCO)CCOCCN1CCOc2cc(N)ccc21 ; Nc1ccc2c(c1)OCCN2CCOCC[N+](CCO)(CCO)CCO]

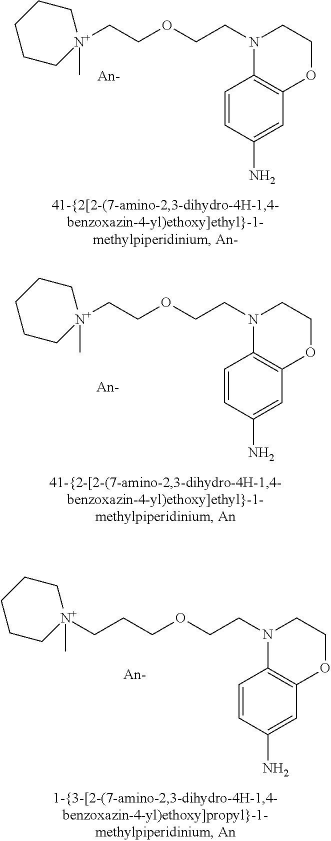 [SMILES: C[N+]1(CCOCCN2CCOc3cc(N)ccc32)CCCCC1 ; C[N+]1(CCOCCN2CCOc3cc(N)ccc32)CCCCC1 ; C[N+]1(CCCOCCN2CCOc3cc(N)ccc32)CCCCC1]

and also addition salts thereof, optical isomers, geometrical isomers and tautomers thereof and/or solvates thereof, An− having the meaning given previously.

Dye Composition

The compounds of formulae (I) as defined above may be used in a composition for dyeing keratin fibres comprising a suitable medium.

The compounds of formula (I) may each be present in the composition in an amount of between 0.001% and 10%, preferably between 0.005% and 6%, by weight approximately of the total weight of the dye composition.

The composition may also comprise, besides said compounds of formula (I), at least one additional oxidation base. These bases may be chosen in particular from para-phenylenediamines, bis(phenyl)alkylenediamines, para-aminophenols, ortho-aminophenols and heterocyclic bases, and addition salts thereof.

Among the para-phenylenediamines, examples that may be mentioned more particularly include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N, N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, 6-(4-aminophenylamino)hexan-1-ol, N-(4-amino-3-methylphenyl)-N-[3-(1H-imidazol-1-yl)propyl]amine and N-(4-aminophenyl)-N-[3-(1H-imidazol-1-yl)propyl]amine, and addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine and 2-[{2-[(4-aminophenyl)amino]ethyl}(2-hydroxyethyl)amino]ethanol, and addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and addition salts thereof with an acid.

Among the para-aminophenols, examples that may be mentioned include para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6-[((5′-amino-2′-hydroxy-3′-methyl)phenyl)methyl]-2-methylphenol and bis[(5′-amino-2′-hydroxy)phenylmethane, and addition salts thereof with an acid.

Among the ortho-aminophenols, examples that may be mentioned include 2-aminophenol, 2-amino-5-methylphenol, 2-amino-6-methylphenol and 5-acetamido-2-aminophenol, and addition salts thereof with an acid.

Among the heterocyclic bases that may be mentioned, for example, are pyridine derivatives, pyrimidine derivatives and pyrazole derivatives.

Among the pyridine derivatives, mention may be made of the compounds described, for example, in patents GB 1 026 978 and GB 1 153 196, such as 2,5-diaminopyridine, 2-(4-methoxyphenyl)amino-3-aminopyridine, 3,4-diaminopyridine, and addition salts thereof with an acid.

Other pyridine oxidation bases that are useful in the present invention are the 3-aminopyrazolo[1,5-a]pyridine oxidation bases or addition salts thereof, described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and addition salts thereof with an acid.

Among the pyridine bases that are of use in the present invention, mention may also be made of the compounds described in patent applications EP 1792903 and EP 1792606 and addition salts thereof.

Among the pyrimidine derivatives, mention may be made of the compounds described, for example, in patents DE 2359399, JP 88-169571, JP 05-63124 and EP 0/770/375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and addition salts thereof, and the tautomeric forms thereof, when a tautomeric equilibrium exists.

Among the pyrazolopyrimidine derivatives, mention may be made of the compounds described, for example, in patent applications EP 0847271, EP 0926149 and EP 1147109 and addition salts thereof.

Among the pyrazole derivatives mention may be made of the compounds described in patents DE 3843892 and DE 4133957 and patent applications WO 94/08969, WO 94/08970, FR-A-2 733 749 and DE 195 43 988, for instance 4,5-diamino1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and addition salts thereof.

As oxidation bases, mention may also be made of the diamino-N,N-dihydropyrazolone derivatives of formula (III) or an addition salt or solvate thereof:

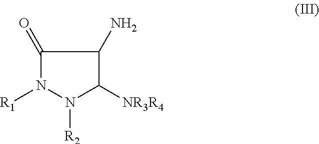

in which:
R₁, R₂, R₃ and R₄, which may be identical or different, represent:
   a linear or branched C₁-C₆ alkyl radical optionally substituted with one or more radicals chosen from the group constituted of a radical OR₅, a radical NR₆R₇, a carboxyl radical, a sulfonic radical, a carboxamido radical CONR₆R₇, a sulfonamido radical SO₂NR₆R₇, a heteroaryl, an aryl optionally substituted with a (C₁-C₄)alkyl, hydroxyl, C₁-C₂ alkoxy, amino or (di)alkyl(C₁-C₂)amino group;
   an aryl radical optionally substituted with one or more (C₁-C₄)alkyl, hydroxyl, C₁-C₂ alkoxy, amino or (di)alkyl(C₁-C₂)amino;
   a 5- or 6-membered heteroaryl radical, optionally substituted with one or more radicals chosen from (C₁-C₄)alkyl and (C₁-C₂)alkoxy;
R₃ and R₄ may also represent a hydrogen atom;
R₅, R₆ and R₇, which may be identical or different, represent a hydrogen atom; a linear or branched C₁-C₄ alkyl radical optionally substituted with one or more radicals chosen from the group constituted of a hydroxyl, a C₁-C₂ alkoxy, a carboxamido CONR₈R₉, a sulfonyl SO₂R₈, an aryl optionally substituted with a (C₁-C₄)alkyl, a hydroxyl, a C₁-C₂ alkoxy, an amino or a (di)(C₁-C₂)alkylamino; an aryl optionally substituted with a (C₁-C₄)alkyl, a hydroxyl, a C₁-C₂ alkoxy, an amino or a (di)(C₁-C₂)alkylamino;
R₆ and R₇, which may be identical or different, may also represent a carboxamido radical CONR₈R₉; a sulfonyl radical SO₂R₈;
R₈ and R₉, which may be identical or different, represent a hydrogen atom; a linear or branched C₁-C₄ alkyl radical optionally substituted with one or more hydroxyl or C₁-C₂ alkoxy;
R₁ and R₂, on the one hand, and R₃ and R₄, on the other hand, may form, with the nitrogen atoms to which they are attached, a saturated or unsaturated 5- to 8-membered heterocycle optionally substituted with one or more radicals chosen from the group constituted of halogen atoms and amino, (di)alkyl(C₁-C₄)amino, hydroxyl, carboxyl, carboxamido and (C₁-C₂)alkoxy radicals, C₁-C₄ alkyl radicals optionally substituted with one or more hydroxyl, amino, (di)alkylamino, alkoxy, carboxyl or sulfonyl radicals;
R₃ and R₄ may also form, together with the nitrogen atom to which they are attached, a 5- or 7-membered heterocycle, the carbon atoms of which may be replaced with an optionally substituted oxygen or nitrogen atom.

These diamino-N,N-dihydropyrazolone derivatives are particularly described in patent application FR 2866338, a particularly preferred derivative being 2,3-diamino-6,7-dihydro-1H,5H-pyrazolo[1,2-a]pyrazol-1-one dimethanesulfonate. Oxidation bases that may also be mentioned include the diamino-N,N-dihydropyrazolone derivatives of formula (IV) or an addition salt or solvate thereof:

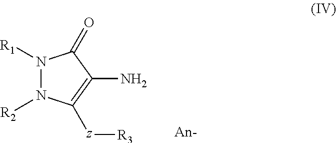

in which:
   z represents independently:
   a single covalent bond,
   a divalent radical chosen from
   an oxygen atom,
   a radical —NR₆, with R₆ representing a hydrogen atom or a C₁-C₆ alkyl radical, or R₆, with R₃, forming, together with the nitrogen atom to which they are attached, a substituted or unsubstituted, saturated or unsaturated, aromatic or non-aromatic, 5- to 8-membered heterocycle, optionally containing one or more other heteroatoms or groups chosen from N, O, S, SO₂ and —CO—, it being possible for the heterocycle to be cationic and/or substituted with a cationic radical,
   a radical —N⁺R₇R₈— with R₇ and R₈ independently representing an alkyl radical; the alkyl radical may be substituted with an OH or an —Oalkyl,
   R₃ represents:
   a hydrogen,
   a C₁-C₁₀ alkyl radical, which is optionally substituted, the alkyl radical possibly being interrupted with a heteroatom or a group chosen from O, N, Si, S, SO and SO₂,
   a C₁-C₁₀ alkyl radical substituted and/or interrupted with a cationic radical,
   a halogen,
   an SO₃H radical,
   a substituted or unsubstituted, saturated, unsaturated or aromatic, 5- to 8-membered ring, optionally containing one or more heteroatoms or groups chosen from N, O, S, SO₂ and —CO—, it being possible for the ring to be cationic and/or substituted with a cationic radical,
   R₁ and R₂, which may be identical or different, represent:
   a linear or branched C₁-C₆ alkyl radical optionally substituted with one or more radicals chosen from a radical OR₅, a radical NR₉R₁₀, a carboxyl radical, a sulfonic radical, a carboxamido radical CONR₉R₁₀, a sulfonamido radical SO₂NR₉R₁₀, a heteroaryl, an aryl optionally substituted with a (C₁-C₄)alkyl, hydroxyl, C₁-C₂ alkoxy, amino or (di)alkyl(C₁-C₂)amino group;
   an aryl radical optionally substituted with one or more (C₁-C₄)alkyl, hydroxyl, C₁-C₂ alkoxy, amino or (di)alkyl(C₁-C₂)amino;
   a 5- or 6-membered heteroaryl radical, optionally substituted with one or more radicals chosen from (C₁-C₄)alkyl monosubstituted or polysubstituted with an OH or an —Oalkyl or (C₁-C₂)alkoxy;
R₁ and R₂ may form, with the nitrogen atoms to which they are attached, a saturated or unsaturated 5- to 8-membered heterocycle optionally substituted with one or more radicals chosen from the group constituted of halogen atoms and amino, (di)alkyl(C₁-C₄)amino, hydroxyl, carboxyl, carboxamido and (C₁-C₂)alkoxy radicals, C₁-C₄ alkyl radicals optionally substituted with one or more hydroxyl, amino, (di)alkylamino, alkoxy, carboxyl or sulfonyl radicals;
An− represents an anion or a group of anions that ensures the electrical neutrality of the compounds of formula (IV),
with the proviso that at least one of the groups Z and R₃ represents a cationic radical.

These derivatives of diamino-N,N-dihydropyrazolone are described in patent application FR 2 927 078.

In general, the concentration of the additional oxidation base(s) ranges from 0.0001% to 20% and preferably from 0.005% to 6% by weight relative to the total weight of the composition.

Couplers

The dye composition according to the invention may contain and preferably contains one or more additional oxidation couplers, other than the compounds of formula (I), that are conventionally used for dyeing keratin fibres. Among these couplers, mention may be made in particular of meta-phenylenediamines, meta-aminophenols, meta-diphenols, naphthalene couplers and heterocyclic couplers, and addition salts thereof.

Examples of additional couplers that may be mentioned include 2-methyl-5-aminophenol, 5-N-(β-hydroxyethyl)amino-2-methylphenol, 6-chloro-2-methyl-5-aminophenol, 3-aminophenol, 2,4-dichloro-3-aminophenol, 5-amino-4-chloro-o-cresol, 1,3-dihydroxybenzene, 1,3-di hydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 1,5-dihydroxynaphthalene, 2,7-naphthalenediol, 1-acetoxy-2-methylnaphthalene, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 2,6-dihydroxy-3-4-dimethylpyridine, 3-amino-2-methylamino-6-methoxypyridine, 1-N-(β-hydroxyethyl)amino-3,4-methylenedioxybenzene, 2,6-bis(β-hydroxyethylamino)toluene and 3-methyl-1-phenyl-5-pyrazolone and addition salts thereof with an acid.

In the dye composition of the present invention, the additional coupler(s), if they are present, generally represent an amount of between 0.001% and 10%, and preferably between 0.005% and 6% by weight approximately relative to the total weight of the composition.

The dye composition in accordance with the invention may also contain one or more direct dyes that may in particular be chosen from nitrobenzene dyes, azo direct dyes and methine direct dyes. These direct dyes may be of nonionic, anionic or cationic nature.

The medium that is suitable for dyeing, also known as the dye support, generally comprises water or a mixture of water and of one or more solvents, for instance C₁-C₄ lower alkanols such as ethanol and isopropanol, polyols, for instance propylene glycol, dipropylene glycol or glycerol, and polyol ethers, for instance dipropylene glycol monomethyl ether.

The solvent(s) are generally present in proportions that may be between 1% and 40% by weight approximately and more preferably between 3% and 30% by weight approximately relative to the total weight of the dye composition.

The dye composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, and in particular anionic, cationic, nonionic and amphoteric polymeric associative thickeners, antioxidants, penetrants, sequestrants, fragrances, buffers, dispersants, conditioning agents, for instance volatile or non-volatile, modified or unmodified silicones, film-forming agents, ceramides, preserving agents and opacifiers.

The above adjuvants are generally present in an amount for each of them of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately. It may be adjusted to the desired value by means of acidifying or basifying agents customarily used in the dyeing of keratin fibres, or alternatively using standard buffer systems.

Among the acidifying agents, examples that may be mentioned include inorganic or organic acids, for instance hydrochloric acid, orthophosphoric acid, sulfuric acid, carboxylic acids, for instance acetic acid, tartaric acid, citric acid and lactic acid, and sulfonic acids.

Among the basifying agents, examples that may be mentioned include aqueous ammonia, alkali metal carbonates, alkanolamines such as monoethanolamine, diethanolamine and triethanolamine, and also derivatives thereof, sodium hydroxide, potassium hydroxide and the compounds of formula (III) below:

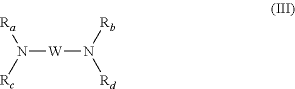

in which W is a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

The composition according to the invention may comprise one or more oxidizing agents.

The oxidizing agents are those conventionally used for the oxidation dyeing of keratin fibres, for example hydrogen peroxide, urea peroxide, alkali metal bromates, persalts such as perborates and persulfates, peracids and oxidase enzymes, among which mention may be made of peroxidases, 2-electron oxidoreductases such as uricases, and 4-electron oxygenases, for instance laccases. Hydrogen peroxide is particularly preferred.

The dye composition with or without oxidizing agent according to the invention may be in various forms, such as in the form of liquids, creams or gels, or in any other form that is suitable for dyeing keratin fibres, and in particular human hair.

It may result from the mixing, at the time of use, of several compositions.

In particular, it results from the mixing of at least two compositions, one comprising at least one compound of formula (I), optionally one or more additional oxidation bases, and optionally one or more additional couplers other than the compounds of formula (I), or salts thereof, and a second composition comprising one or more oxidizing agents as described previously.

The composition of the invention is thus applied to the hair either in unmodified form or in the presence of one or more oxidizing agents, for the dyeing of keratin fibres.

The process of the present invention is a process in which the composition according to the present invention as defined previously is applied to the fibres, either alone or in the presence of an oxidizing agent, for a time that is sufficient to develop the desired colouring. The colour may be developed at acidic, neutral or alkaline pH, and the oxidizing agent may be added to the composition of the invention just at the time of use, or it may be used starting from an oxidizing composition which comprises it and which is applied simultaneously with or sequentially to the composition of the invention.

According to a particular embodiment, the composition is free of oxidizing agent and is mixed, preferably at the time of use, with a composition containing, in a medium that is suitable for dyeing, one or more oxidizing agents, these oxidizing agents being present in an amount sufficient to develop a colouring. The mixture obtained is then applied to the keratin fibres. After a leave-on time of approximately 3 to 50 minutes, preferably approximately 5 to 30 minutes, the keratin fibres are rinsed, washed with shampoo, rinsed again and then dried.

The oxidizing agents are those indicated above.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably varies between 3 and 12 approximately and more preferably still between 5 and 11. It may be adjusted to the desired value by means of acidifying or basifying agents usually used in the dyeing of keratin fibres and as defined previously.

The ready-to-use composition that is finally applied to the keratin fibres may be in various forms, such as in the form of liquids, creams or gels or in any other form that is suitable for dyeing keratin fibres, and in particular human hair.

Another subject of the invention is a dyeing “kit” or multi-compartment device in which a first compartment contains the dye composition devoid of oxidizing agent of the present invention defined above, comprising one or more oxidation bases chosen from the compounds of formula (I) or addition salts thereof with an acid, and a second compartment contains one or more oxidizing agents.

These devices may be equipped with a means for dispensing the desired mixture on the hair, such as the devices described in patent FR-2 586 913 in the name of the Applicant.

Preparation of the Compounds of Formula (I)

The synthesis of the compound of formula (I) may be performed, for example, according to the following procedures described hereinafter.

According to one particular embodiment, the synthesis of the cationic compounds of formula (I) may be carried out using the structural distributor “A”, prepared according to the following scheme:

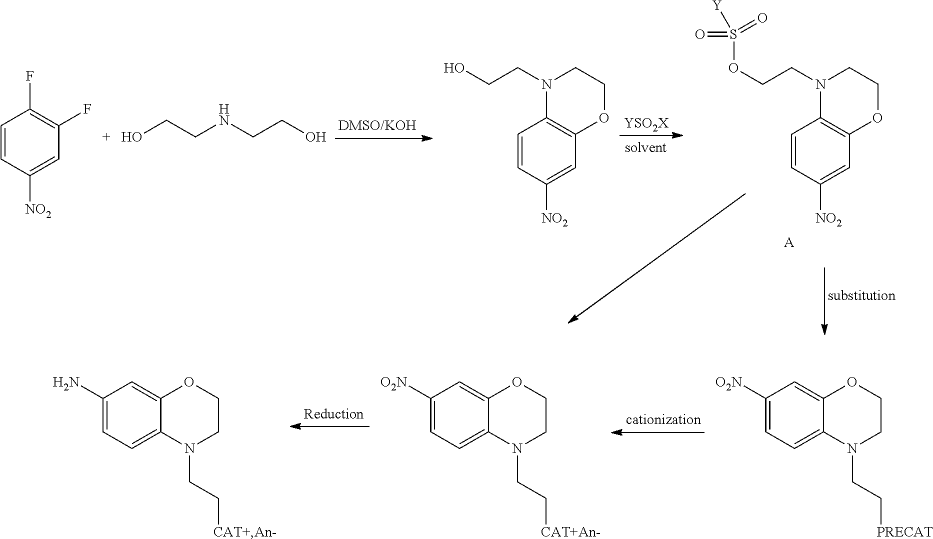

The structural distributor “A” is then used according to the reaction scheme below to give the compounds of formula (I).

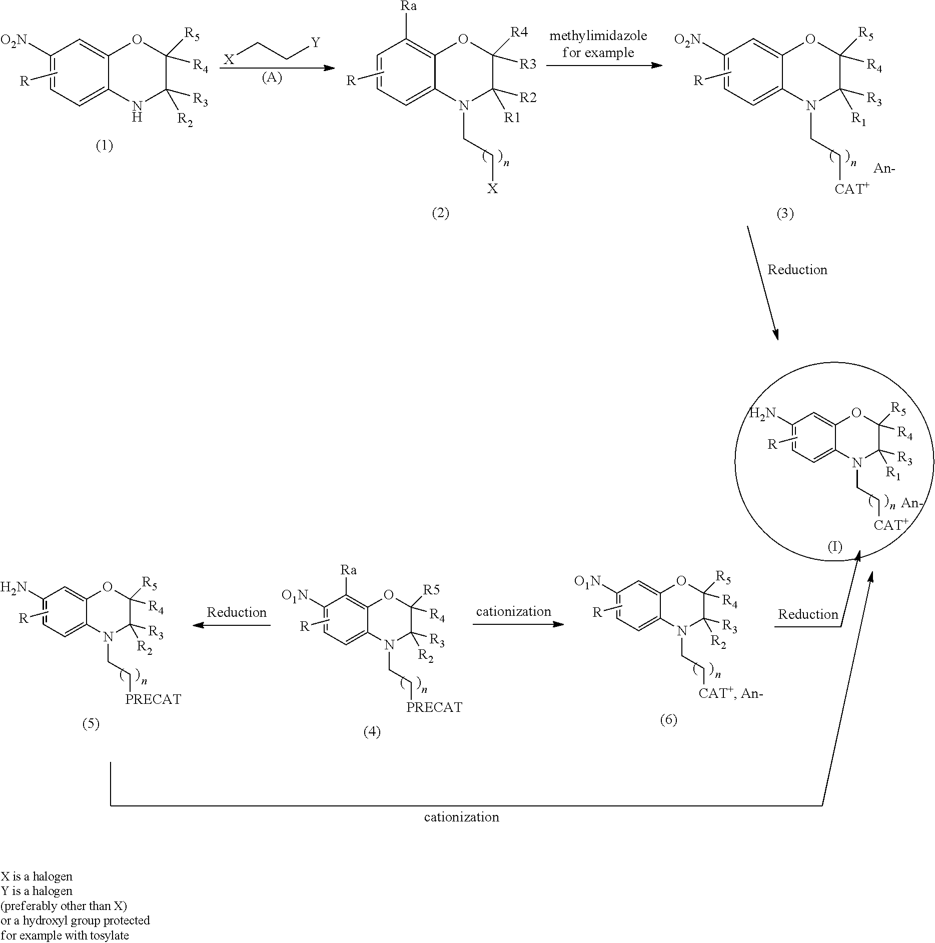

According to one particular embodiment, the synthesis of the compounds of formula (I), in which L represents a group

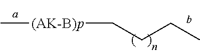

as defined previously, can be carried out according to the following schemes:

When p=0

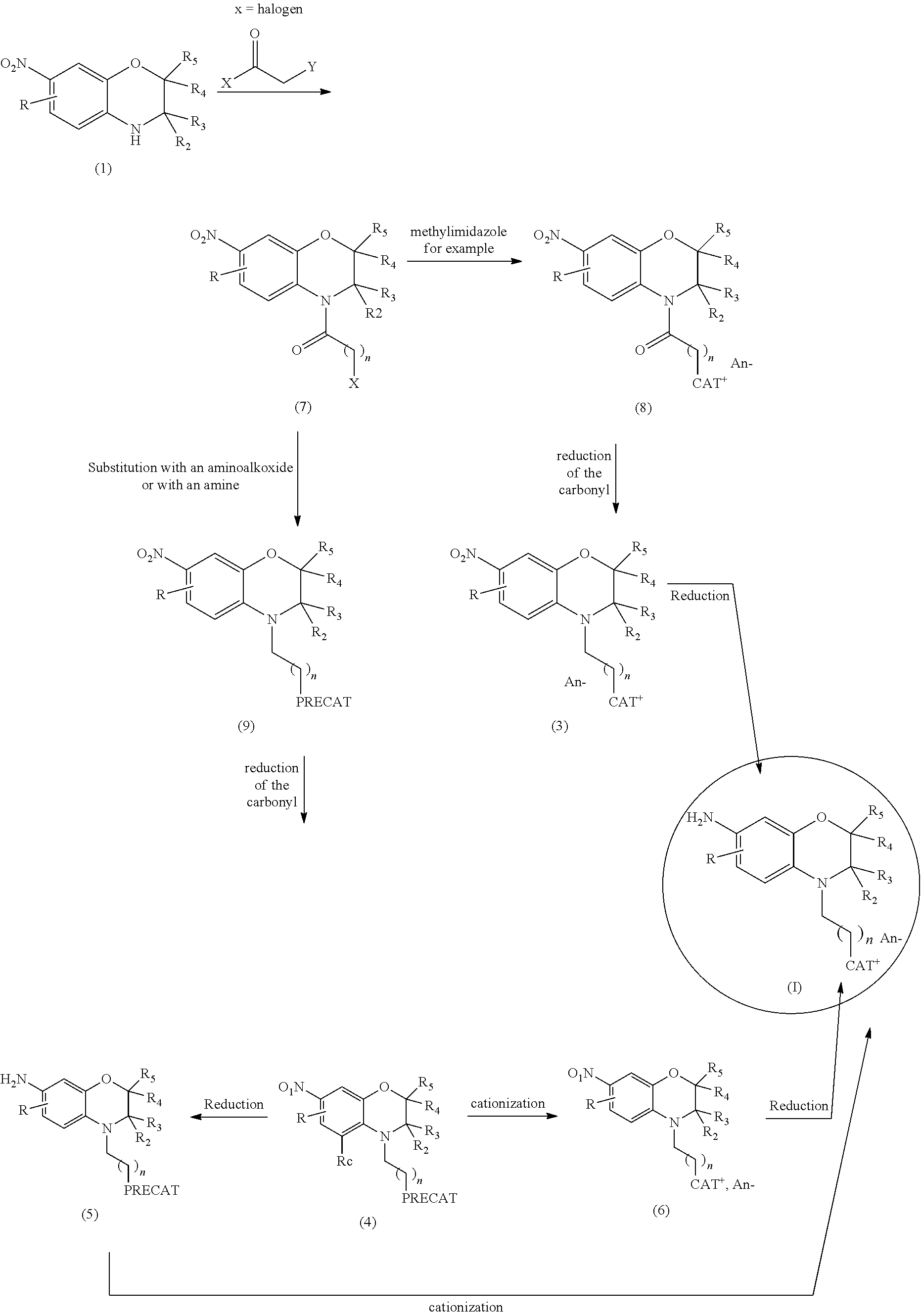

When p=1 or 2

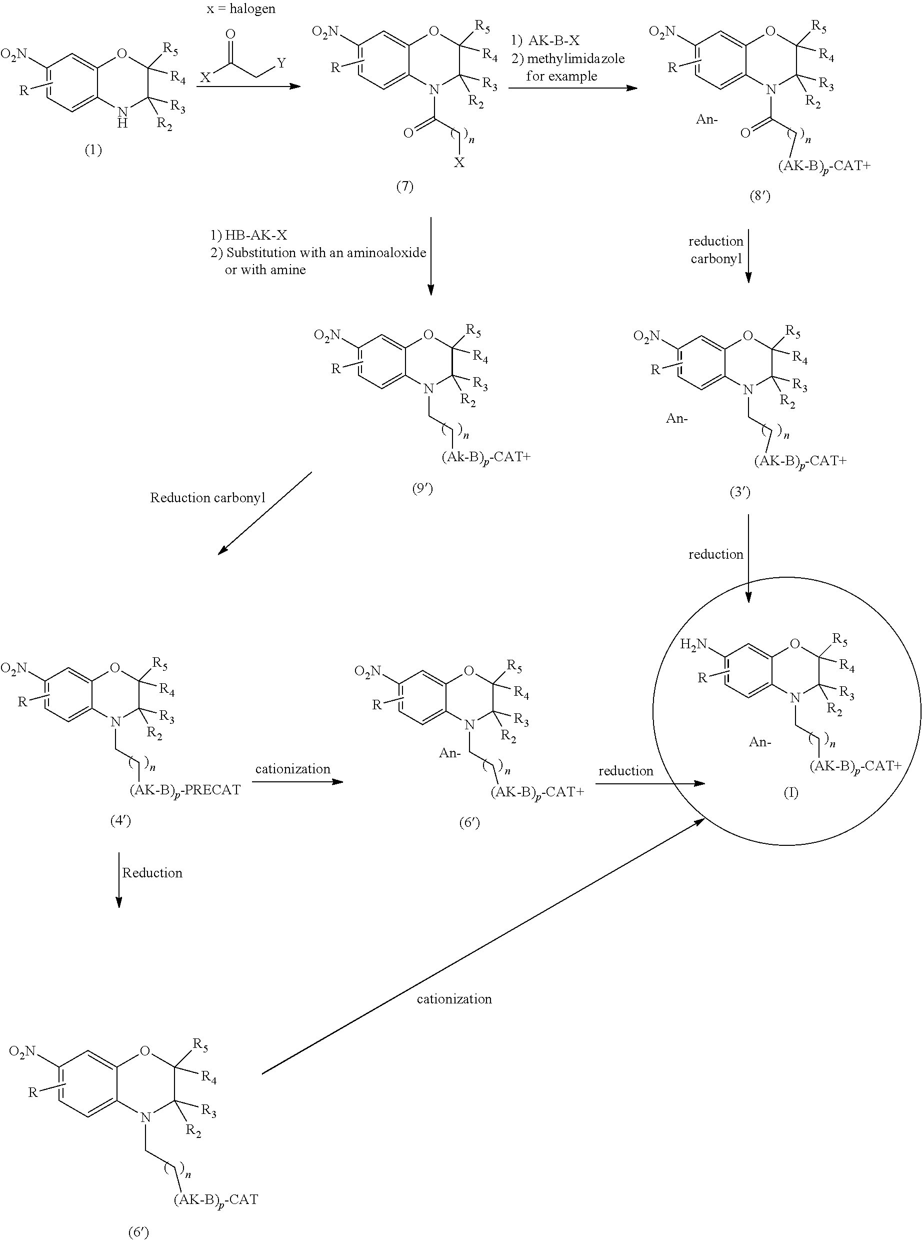

Or

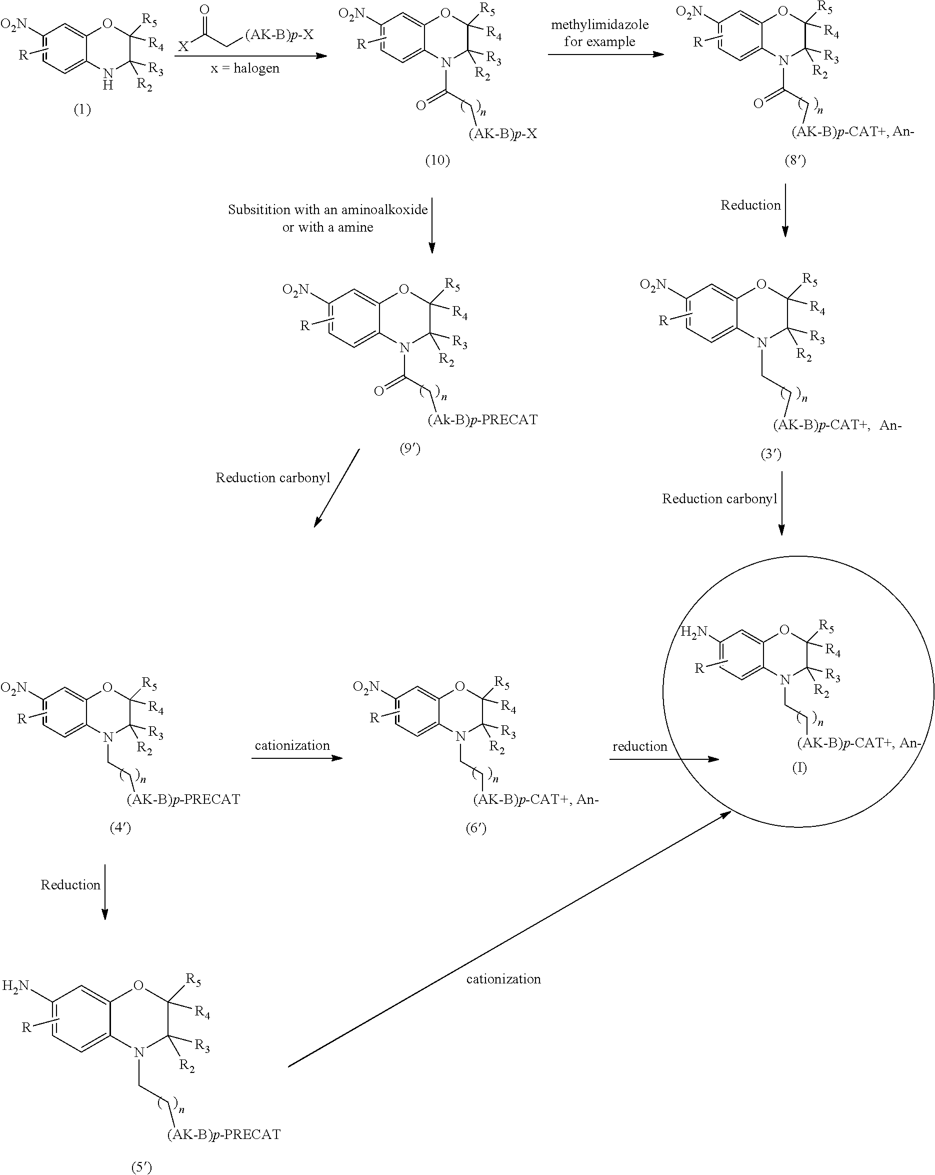

CAT+ being a cationic radical as defined previously, the electrical neutrality being ensured by An− as defined previously,

PRECAT being a cationizable radical which produces the CAT+ radical after cationization.

The first step is an acylation reaction or an alkylation reaction of the 7-nitro-1,2,3,4-tetrahydroquinoline (1) under conventional conditions, for example by reaction with a haloacetyl halide or a derivative (A) in a polar solvent of alcohol, alkyl acetate, THF, dioxane, etc. type, at a temperature of between 0° C. and 100° C. Depending on the nature of the haloacetyl, the compound (7) or (10) is obtained, the reaction with the compound (A) resulting in the intermediate (2).

Depending on the nature of the amine subsequently used, either the cationic nitrogenous compound (3), (8) or (8′) is directly produced, or it is necessary to substitute the leaving group of the compound (2) or (7) or (10) with an amine or an aminoalkoxide so as to produce the derivative (4) or (9) or (9′). The carbonyl functions of the compounds (7), (9) and (9′) are then reduced under the conventional conditions, for example with BH₃ in a polar or non-polar solvent, for instance THF, so as to give the derivative (4) or (4′).

The derivatives (4) and (4′) are either cationized so as to give (6) or (6′) or their nitro function is reduced so as to give the compound (5) or (5′).

The compounds of the invention are then reduced or cationized as indicated in the reaction schemes.

The reduction of the nitro group is performed under standard conditions, for example by performing a hydrogenation reaction under heterogeneous catalysis in the presence of Pd/C, Pd(II)/C, Ni/Ra, etc., or alternatively by performing a reduction reaction with a metal, for example with zinc, iron, tin, etc. (see Advanced Organic Chemistry, 3rd Edition, J. March, 1985, Wiley Interscience and Reduction in Organic Chemistry, M. Hudlicky, 1983, Ellis Horwood Series Chemical Science).

The cationization is carried out by reaction with at least one equivalent of alkyl halide, methyl sulfate or alkyl carbonate in a solvent such as THF, acetonitrile, dioxane or ethyl acetate for 15 min to 24 h at a temperature ranging from 15° C. to the reflux temperature of the solvent so as to give the cationic nitrogenous compounds.

The examples which follow serve to illustrate the invention without, however, exhibiting a limiting nature.

### EXAMPLES

### Synthesis of the structural distributor A=2-(7-nitro-2,3-dihydro-4H-1,4-benzoxazin-4-yl)ethyl methanesulfonate

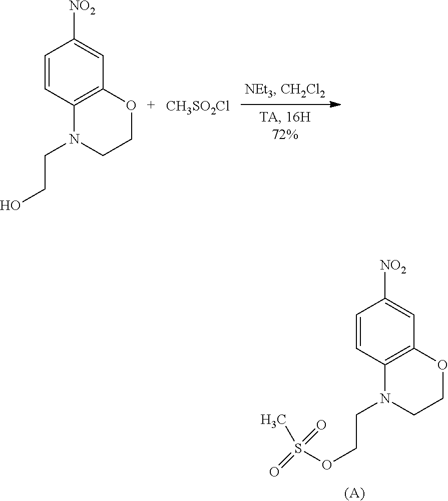

100 ml of dichloromethane, 6.7 g (0.030 mol) of 2-(7-nitro-2,3-dihydro-4H-1,4-benzoxazin-4-yl)ethanol and 6.8 g (0.06 mol) of mesyl chloride are successively placed in a 100 ml three-necked flask equipped with a thermometer, a condenser, a bubbler and a dropping funnel, with magnetic stirring. 6 g (0.060 mol) of triethylamine are added dropwise to this solution. This clear solution is stirred at ambient temperature for 16 hours.

The organic phase is washed with water and dried over anhydrous magnesium sulfate, and the solvent is evaporated off to give a crude product, which, after purification by recrystallization from ethyl acetate, gives 6.8 g (72% yield) of expected compound A in the form of a yellow powder.

The NMR (¹H 400 MHz and ¹³C 100.61 MHz DMSO-d₆) and mass spectrometry analyses are in accordance with the expected structure.

The mass spectrometry analysis confirms the expected compound C11H14N2O6. The quasi-molecular ions [M+H]+, [M+Na]+, [M−H]− of the expected molecule are mainly detected.

### Example 1: Synthesis of 1-[2-(7-amino-2,3-dihydro-4H-1,4-benzoxazin-4-yl)ethyl]-3-methyl-1H-imidazol-3-ium methanesulfonate hydrochloride

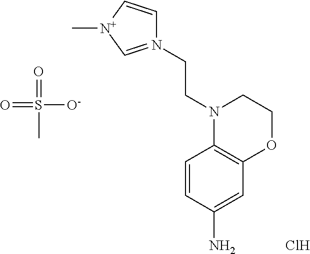

### Synthesis of 3-methyl-1-[2-(7-nitro-2,3-dihydro-4H-1,4-benzoxazin-4-yl)ethyl]-1H-imidazol-3-ium methanesulfonate

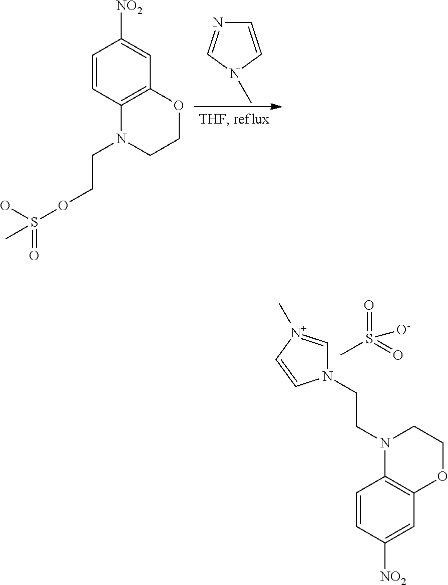

300 ml of tetrahydrofuran, 2 g (0.066 mol) of structural distributor “A” 2-(7-nitro-2,3-dihydro-4H-1,4-benzoxazin-4-yl)ethyl methanesulfonate and then 1.58 ml (0.07 mol) of methylimidazole are successively added, dropwise, to a 50 ml three-necked flask equipped with a thermometer, a condenser, a bubbler and a dropping funnel, with magnetic stirring. The homogeneous medium is heated for 30 minutes. The precipitate formed is isolated by filtration after cooling to ambient temperature.

The solid formed is recovered and dried under vacuum at 30° C. in the presence of desiccant, and gives 2.15 g (84.6% yield) of expected compound in the form of a yellow powder.

The NMR analyses (1D 1H and 2D 1H/13C NMR spectra of HMBC type) are in accordance with the expected structure.

### Synthesis of 1-[2-(7-amino-2,3-dihydro-4H-1,4-benzoxazin-4-yl)ethyl]-3-methyl-1H-imidazol-3-ium methanesulfonate hydrochloride

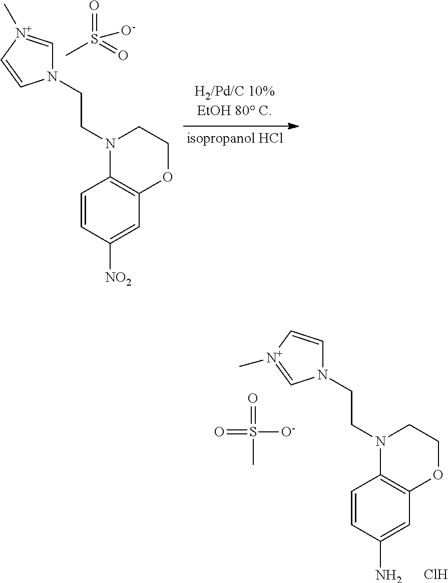

A solution resulting from 5 g (0.0165 mol) of 3-methyl-1-[2-(7-nitro-2,3-dihydro-4H-1,4-benzoxazin-4-yl)ethyl]-1H-imidazol-3-ium methanesulfonate in 1000 ml of absolute ethanol is introduced at a flow rate of 3 ml per minute onto a cartridge of palladium catalyst at 80° C. under a pressure of 50 bar in the H-Cube system in the presence of hydrogen.

At the machine outlet, the solution obtained is added to a solution of 75 ml of 6N hydrochloric isopropanol under argon.

The light-grey solid formed is recovered by filtration and is dried under vacuum at 30° C. in the presence of desiccant, to give 4.4 g (84.3% yield) of expected compound in the form of a light-grey powder.

The NMR analyses (1D 1H and 2D 1H/13C NMR spectra of HMBC type) are in accordance with the expected structure.

### Example 2: Dye Compositions

The following dye compositions were prepared:

| | Example | | | | |
|---|---|---|---|---|---|
| | A | B | C | D | E |
| 1-[2-(7-Amino-2,3- | 10⁻³ mol | 10⁻³ mol | 10⁻³ mol | 10⁻³ mol | 10⁻³ mol |
| dihydro-4H-1,4- | | | | | |
| benzoxazin-4-yl)ethyl]-3- | | | | | |
| methyl-1H-imidazol-3-ium | | | | | |
| methanesulfonate | | | | | |
| hydrochloride | | | | | |
| 2-(2,4- | 10⁻³ mol | | | | |
| diaminophenoxy)ethanol | | | | | |
| hydrochloride | | | | | |
| 5-Amino-2-methylphenol | | 10⁻³ mol | | | |
| 2-METHYL-5- | | | 10⁻³ mol | | |
| HYDROXYETHYLAMINO | | | | | |
| PHENOL | | | | | |
| 6- | | | | 10⁻³ mol | |
| HYDROXYBENZOMORPHOLINE | | | | | |
| 2-AMINO-3- | | | | | 10⁻³ mol |
| HYDROXYPYRIDINE | | | | | |
| Dye support (1) | (*) | (*) | (*) | (*) | (*) |
| Demineralized water qs | 100 g | 100 g | 100 g | 100 g | 100 g |
| Shade observed | Blue | Chestnut | light | dark | dark |
| | green | Brown | brown | brown | purple |
| | | | | | ash brown |

(*): dye support (1) pH 9.5

| | | |
|---|---|---|
| 96° ethyl alcohol | 20.8 | g |
| 35% aqueous sodium metabisulfite solution | 0.23 | g AM |
| Pentasodium salt of diethylenetriaminepentaacetic acid as an aqueous | 0.48 | g AM |
| 40% solution | | |
| C₈-C₁₀ alkyl polyglucoside as an aqueous 60% solution | 3.6 | g AM |
| Benzyl alcohol | 2.0 | g |
| Polyethylene glycol containing 8 units of ethylene oxide | 3.0 | g |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After a leave-in time of 30 minutes, the locks are rinsed, washed with a standard shampoo, rinsed again and then dried, to give the shades mentioned.

1. Compound of formula (I) below, addition salts thereof, optical isomers, geometrical isomers and tautomers thereof and/or solvates thereof: 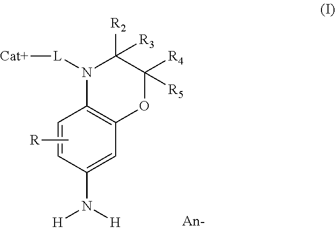
in which
R represents a hydrogen atom or a C₁-C₄ alkyl or C₁-C₄ hydroxyalkyl radical; preferably, R represents a hydrogen atom,
R₂, R₃, R₄ and R₅ independently represent:
a hydrogen atom,
a linear C₁-C₄ or branched C₃-C₄ alkyl radical, optionally substituted with a hydroxyl radical,
L represents a linear or branched, saturated C₁-C₃₀ hydrocarbon-based chain, optionally substituted with one or more hydroxyl radicals, optionally interrupted with one or more non-adjacent oxygen atoms or with one or more non-adjacent radicals NR′, R′ denoting a hydrogen atom or a C₁-C₄ alkyl radical,
CAT+ represents a cationic radical chosen from:
a cationic heterocyclic radical comprising 5 to 8 ring members containing at least one heteroatom chosen from O, N and S, optionally substituted with one or more radicals, which may be identical or different, chosen from linear or branched C₁-C₄ alkyl radicals or linear or branched C₁-C₄ hydroxyalkyl radicals,
a tri(hydroxy)(C₁-C₄)alkylammonium radical,
a non-cationic heterocycle comprising 5 or 6 ring members containing a heteroatom chosen from O and N, such as pyrrolidine, substituted with a cationic radical chosen from:
imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums, benzoxazoliums and piperidiniums, which are optionally substituted with one or more identical or different radicals chosen from linear or branched C₁-C₄ alkyl radicals and linear or branched C₁-C₄ hydroxyalkyl radicals, or
a tri(hydroxy)(C₁-C₄)alkylammonium radical,
An− represents an anion or a mixture of anions which are organic or inorganic and are cosmetically acceptable.

2. Composition according to claim 1, characterized in that L represents a linear or branched, saturated C₁-C₂₀, preferably C₁-C₁₀, hydrocarbon-based chain, optionally substituted with one or more hydroxyl radicals, optionally interrupted with one or two non-adjacent oxygen atoms or non-adjacent radicals NR′, R′ denoting a hydrogen atom or a C₁-C₄ alkyl radical.

3. Compound according to either of the preceding claims, characterized in that L represents a group: 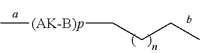
in which:
B represents an oxygen atom or a radical NR′ in which R′ represents a hydrogen atom or a C₁-C₄ alkyl radical,
n represents an integer between 1 and 5, preferably n=1 or 2,
AK represents a linear or branched, saturated C₁-C₁₀ hydrocarbon-based chain which is optionally substituted with one or more hydroxyl radicals,
p=0, 1 or 2, when p is equal to 2, the radicals B respectively AK are identical or different, and
the bond a links the group L to the cationic radical CAT+ and the bond b links the group L to the nitrogen atom of the oxazine ring.

4. Compound according to claim 3, characterized in that p=0 or 1.

5. Compound according to claim 3 or 4, characterized in that AK represents a linear or branched, saturated C₁-C₆ hydrocarbon-based chain, optionally substituted with one or more hydroxyl radicals, preferably a linear, saturated C₁-C₄ hydrocarbon-based chain.

6. Compound according to one of the preceding claims, characterized in that R′ represents H or CH₃, preferably a hydrogen atom.

7. Compound according to one of the preceding claims, characterized in that L represents a linear, saturated C₁-C₆ hydrocarbon-based chain.

8. Compound according to one of claims 1 to 6, characterized in that L represents a linear C₂-C₁₀ alkyl radical, interrupted with an oxygen atom and/or with an —NH group.

9. Compound according to one of the preceding claims, characterized in that the cationic heterocyclic radical comprising 5 to 8 ring members containing at least one heteroatom chosen from O, N and S is chosen from imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums, benzoxazoliums and piperidiniums.

10. Compound according to one of the preceding claims, characterized in that the tri(hydroxy)(C₁-C₄)alkylammonium radical is chosen from trimethylammonium, triethylammonium, dimethylethylammonium, diethylmethylammonium, diisopropylmethylammonium, diethylpropylammonium, 2-hydroxyethyldiethylammonium, di-beta-hydroxyethylmethylammonium, tri-beta-hydroxyethylammonium, dimethylhydroxyethylammonium, 2-hydroxyethyldimethylammonium and 2-hydroxyethylmethylethylammonium.

11. Compound according to one of the preceding claims, characterized in that CAT+ represents a cationic radical chosen from:
a cationic heterocyclic radical chosen from imidazoliums, piperaziniums, pyrrolidiniums, morpholiniums and piperidiniums which are optionally substituted with one or more identical or different radicals chosen from linear or branched C₁-C₄ alkyl radicals and linear or branched C₁-C₄ hydroxyalkyl radicals,
a tri(hydroxy)(C₁-C₄)alkylammonium radical chosen from trimethylammonium, triethylammonium, dimethylethylammonium, diethylmethylammonium, diisopropylmethylammonium, diethylpropylammonium, 2-hydroxyethyldimethylammonium, diethylhydroxyethylammonium and 2-hydroxyethyldiethylammonium,
a non-cationic heterocycle comprising 5 or 6 ring members containing a heteroatom chosen from O and N, such as pyrrolidine for example, substituted with a tri(hydroxy)(C₁-C₄)alkylammonium radical, for instance trimethylammonium, triethylammonium, dimethylethylammonium, diethylmethylammonium, diisopropylmethylammonium, diethylpropylammonium, 2-hydroxyethyldiethylammonium, 2-hydroxyethylmethylethylammonium, di-beta-hydroxyethylmethylammonium, tri-beta-hydroxyethylammonium, dimethylhydroxyethylammonium or 2-hydroxyethyldimethylammonium, in particular trimethylammonium or 2-hydroxyethylmethylethylammonium.

12. Compound according to one of the preceding claims, characterized in that CAT+ represents a cationic radical chosen from:
a cationic heterocyclic radical chosen from imidazoliums, piperaziniums, pyrrolidiniums, morpholiniums and piperidiniums which are optionally substituted with one or more identical or different radicals chosen from linear or branched C₁-C₄ alkyl radicals and linear C₁-C₄ hydroxyalkyl radicals,
a radical chosen from trimethylammonium, triethylammonium, dimethylethylammonium, diethylmethylammonium, diisopropylmethylammonium, 2-hydroxyethyldimethylammonium, 2-hydroxyethylmethylethylammonium, di-beta-hydroxyethylmethylammonium, tri-beta-hydroxyethylammonium, dimethylhydroxyethylammonium, and 2-hydroxyethyldimethylammonium, in particular trimethylammonium and 2-hydroxyethylmethylethylammonium,
a pyrrolidine radical substituted with a trimethylammonium radical.

13. Compound according to one of the preceding claims, characterized in that R, R₂, R₃, R₄ and R₅ independently represent a hydrogen atom or a linear C₁-C₄ or branched C₃-C₄ alkyl radical.

14. Compound according to one of the preceding claims, characterized in that R, R₂, R₃, R₄ and R₅ represent a hydrogen atom.

15. Compound of formula (I) according to one of the preceding claims, characterized in that it is chosen from the following compounds: 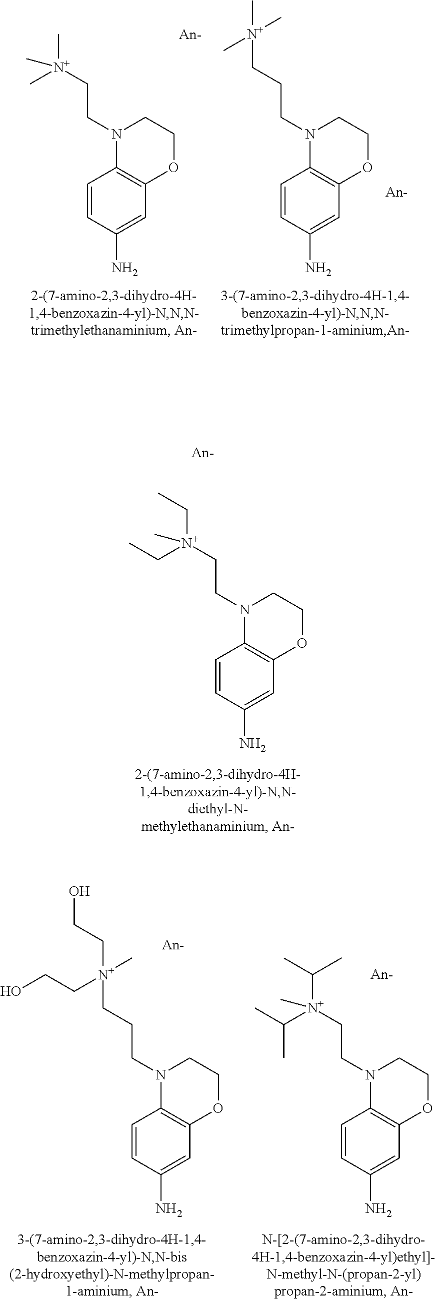 [SMILES: C[N+](C)(C)CCN1CCOc2cc(N)ccc21 ; C[N+](C)(C)CCCN1CCOc2cc(N)ccc21 ; CC[N+](C)(CC)CCN1CCOc2cc(N)ccc21 ; C[N+](CCO)(CCO)CCCN1CCOc2cc(N)ccc21 ; CC(C)[N+](C)(CCN1CCOc2cc(N)ccc21)C(C)C] 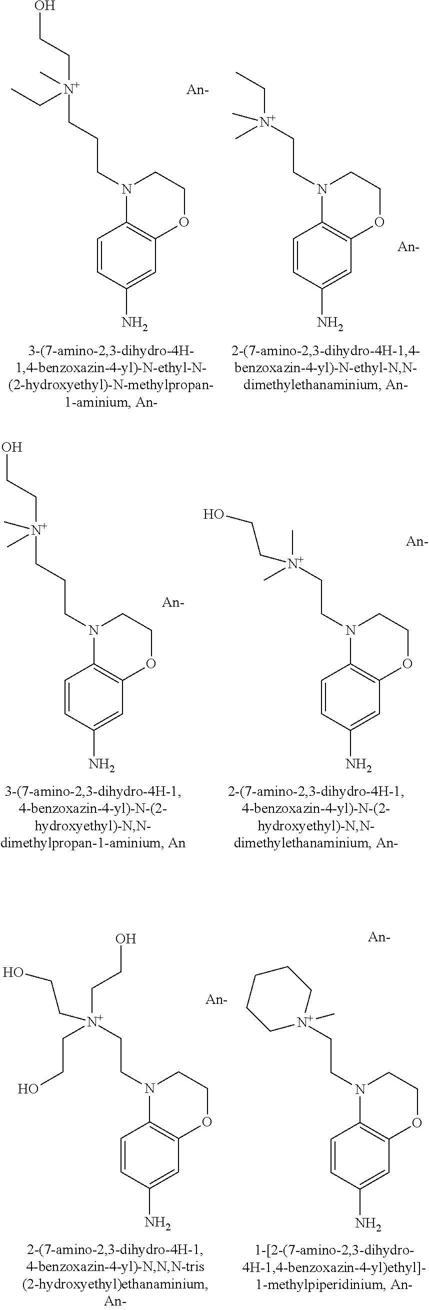 [SMILES: CC[N+](C)(CCO)CCCN1CCOc2cc(N)ccc21 ; CC[N+](C)(C)CCN1CCOc2cc(N)ccc21 ; C[N+](C)(CCO)CCCN1CCOc2cc(N)ccc21 ; C[N+](C)(CCO)CCN1CCOc2cc(N)ccc21 ; Nc1ccc2c(c1)OCCN2CC[N+](CCO)(CCO)CCO ; C[N+]1(CCN2CCOc3cc(N)ccc32)CCCCC1] 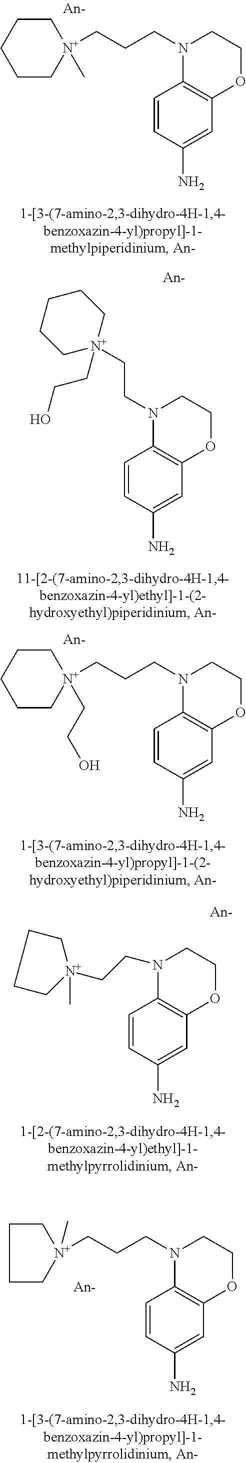 [SMILES: C[N+]1(CCCN2CCOc3cc(N)ccc32)CCCCC1 ; Nc1ccc2c(c1)OCCN2CC[N+]1(CCO)CCCCC1 ; Nc1ccc2c(c1)OCCN2CCC[N+]1(CCO)CCCCC1 ; C[N+]1(CCN2CCOc3cc(N)ccc32)CCCC1 ; C[N+]1(CCCN2CCOc3cc(N)ccc32)CCCC1] 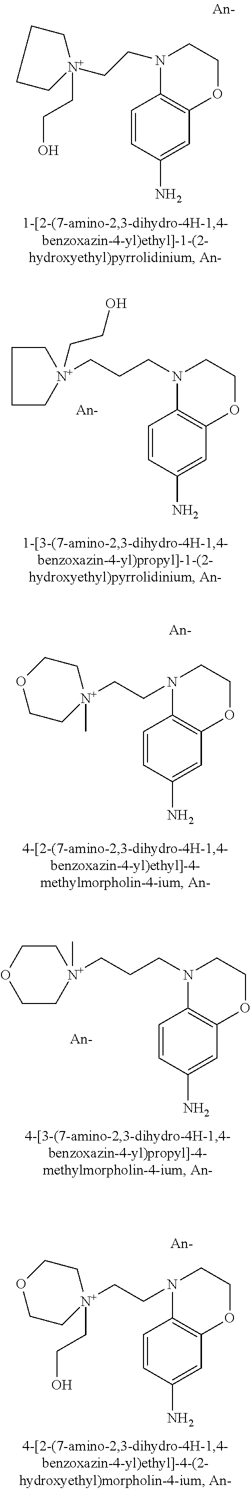 [SMILES: Nc1ccc2c(c1)OCCN2CC[N+]1(CCO)CCCC1 ; Nc1ccc2c(c1)OCCN2CCC[N+]1(CCO)CCCC1 ; C[N+]1(CCN2CCOc3cc(N)ccc32)CCOCC1 ; C[N+]1(CCCN2CCOc3cc(N)ccc32)CCOCC1 ; Nc1ccc2c(c1)OCCN2CC[N+]1(CCO)CCOCC1] 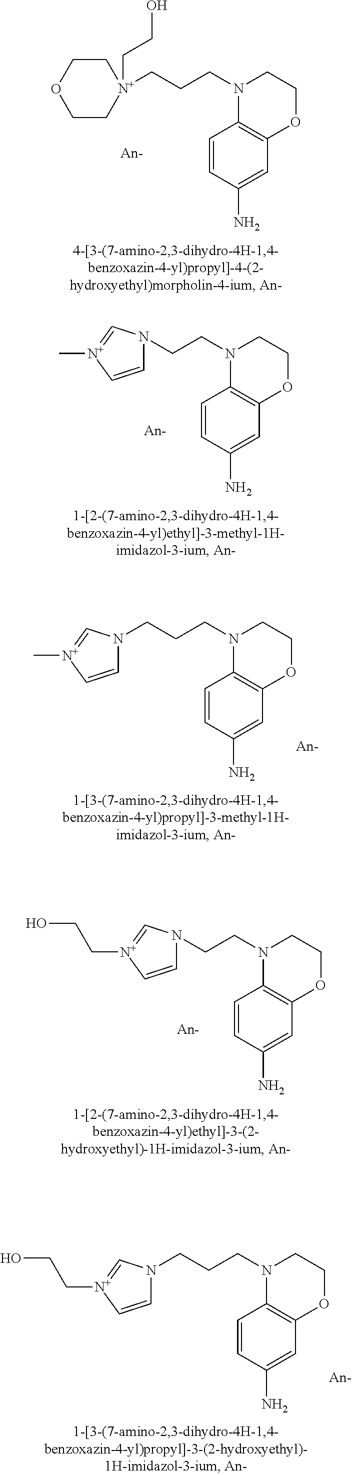 [SMILES: Nc1ccc2c(c1)OCCN2CCC[N+]1(CCO)CCOCC1 ; C[n+]1ccn(CCN2CCOc3cc(N)ccc32)c1 ; C[n+]1ccn(CCCN2CCOc3cc(N)ccc32)c1 ; Nc1ccc2c(c1)OCCN2CCn1cc[n+](CCO)c1 ; Nc1ccc2c(c1)OCCN2CCCn1cc[n+](CCO)c1] 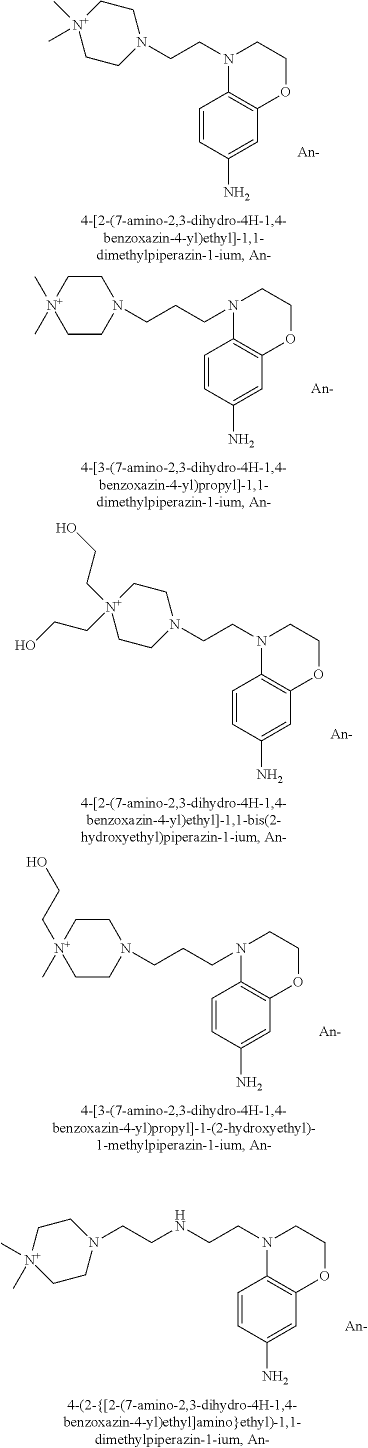 [SMILES: C[N+]1(C)CCN(CCN2CCOc3cc(N)ccc32)CC1 ; C[N+]1(C)CCN(CCCN2CCOc3cc(N)ccc32)CC1 ; Nc1ccc2c(c1)OCCN2CCN1CC[N+](CCO)(CCO)CC1 ; C[N+]1(CCO)CCN(CCCN2CCOc3cc(N)ccc32)CC1 ; C[N+]1(C)CCN(CCNCCN2CCOc3cc(N)ccc32)CC1] 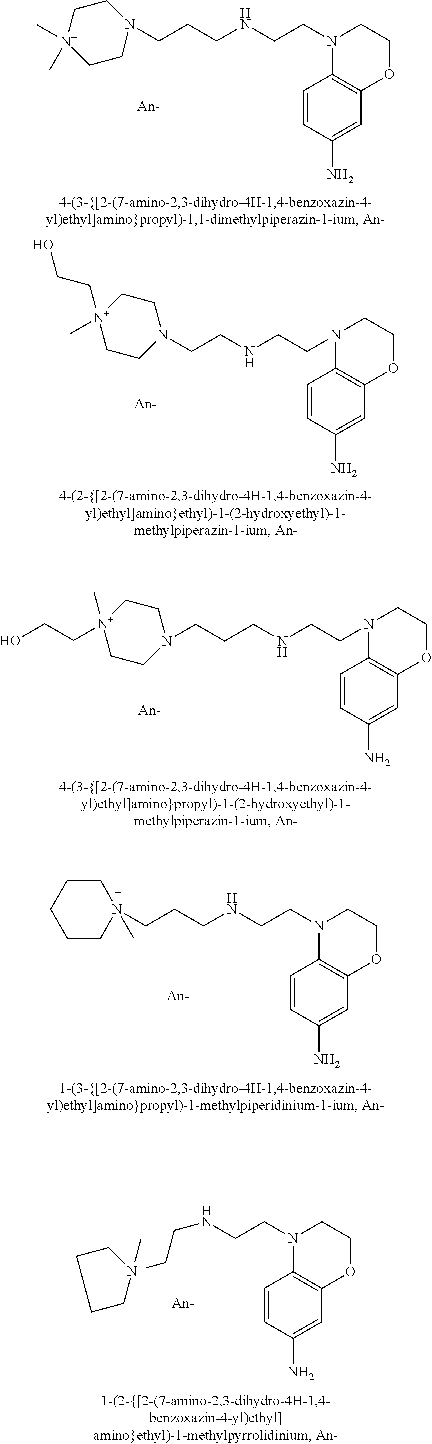 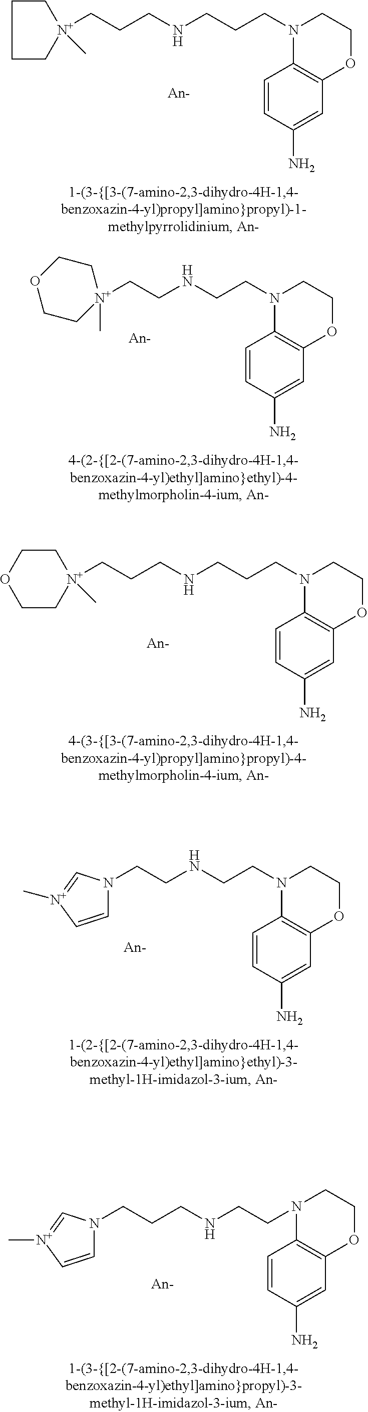 [SMILES: C[N+]1(CCCNCCCN2CCOc3cc(N)ccc32)CCCC1 ; C[N+]1(CCNCCN2CCOc3cc(N)ccc32)CCOCC1 ; C[N+]1(CCCNCCCN2CCOc3cc(N)ccc32)CCOCC1 ; C[n+]1ccn(CCNCCN2CCOc3cc(N)ccc32)c1 ; C[n+]1ccn(CCCNCCN2CCOc3cc(N)ccc32)c1] 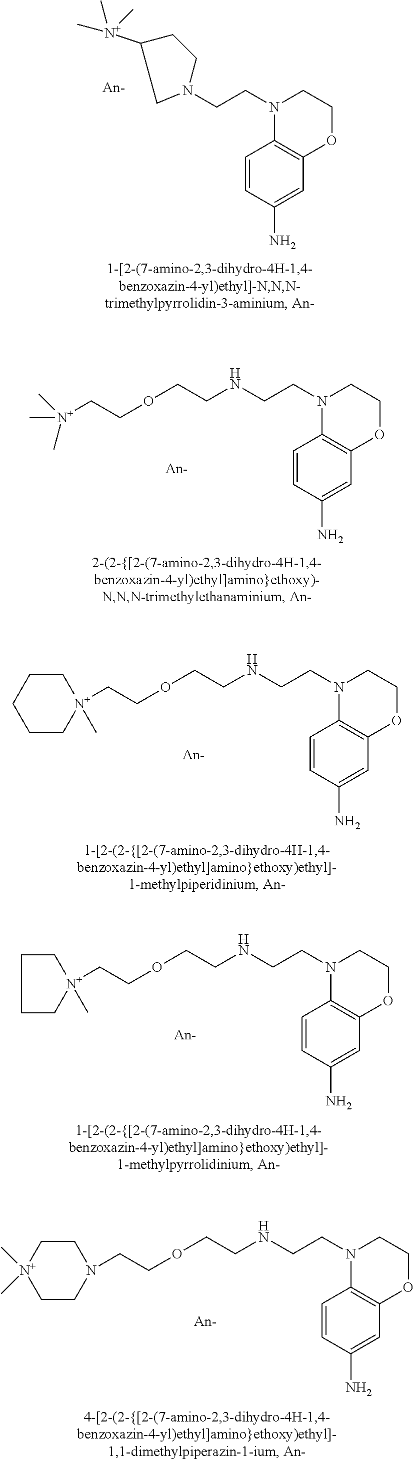 [SMILES: C[N+](C)(C)C1CCN(CCN2CCOc3cc(N)ccc32)C1 ; C[N+](C)(C)CCOCCNCCN1CCOc2cc(N)ccc21 ; C[N+]1(CCOCCNCCN2CCOc3cc(N)ccc32)CCCCC1 ; C[N+]1(CCOCCNCCN2CCOc3cc(N)ccc32)CCCC1 ; C[N+]1(C)CCN(CCOCCNCCN2CCOc3cc(N)ccc32)CC1] 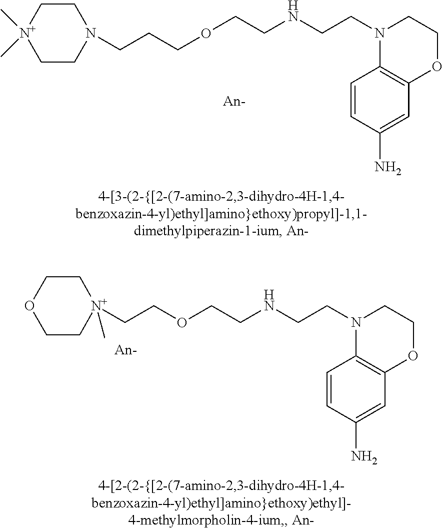 [SMILES: C[N+]1(C)CCN(CCCOCCNCCN2CCOc3cc(N)ccc32)CC1 ; C[N+]1(CCOCCNCCN2CCOc3cc(N)ccc32)CCOCC1] 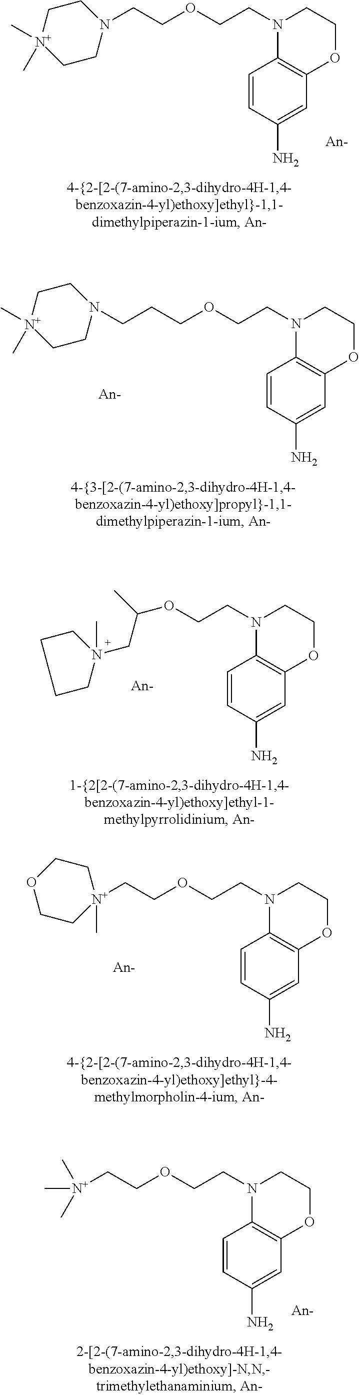 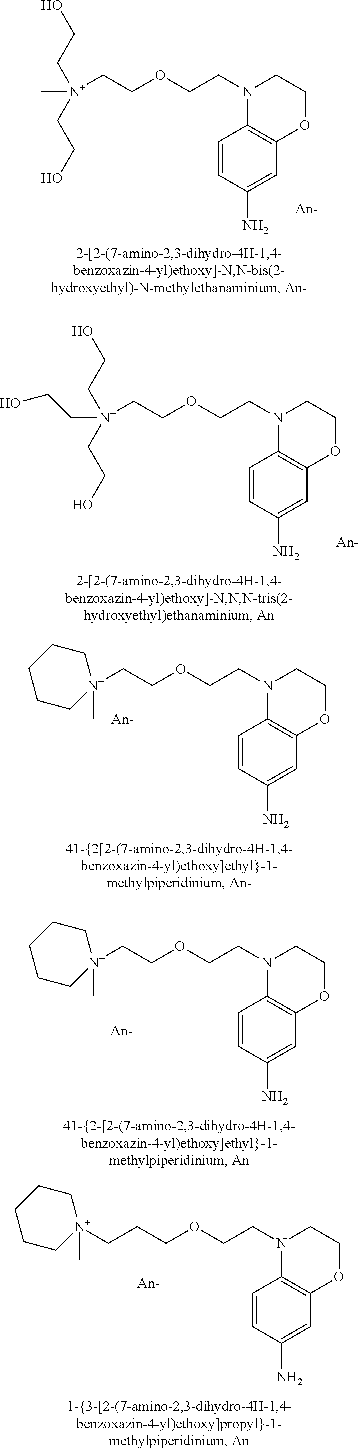 [SMILES: C[N+](CCO)(CCO)CCOCCN1CCOc2cc(N)ccc21 ; Nc1ccc2c(c1)OCCN2CCOCC[N+](CCO)(CCO)CCO ; C[N+]1(CCOCCN2CCOc3cc(N)ccc32)CCCCC1 ; C[N+]1(CCOCCN2CCOc3cc(N)ccc32)CCCCC1 ; C[N+]1(CCCOCCN2CCOc3cc(N)ccc32)CCCCC1]
and also addition salts thereof, optical isomers, geometrical isomers and tautomers thereof and/or solvates thereof.

16. Use of a compound chosen from the compounds of formula (I) as defined in one of claims 1 to 15, for dyeing keratin fibres, in particular human keratin fibres such as the hair.

17. Process for dyeing keratin fibres, in particular the hair, which consists in applying to said fibres a composition comprising a compound of formula (I) according to one of claims 1 to 15.

18. Composition, in particular cosmetic composition, comprising at least one benzoxazine-based compound of formula (I) according to one of claims 1 to 15.
